(19) United States
(12) Patent Application Publication
(10) Pub. No.: US 2025/0277196 A1
(43) Pub. Date: Sep. 4, 2025

(54) EPSPS MUTANTS AND METHOD OF ITS USES

(71) Applicant: CROPEDIT BIOTECHNOLOGY INC., Beijing (CN)

(72) Inventors: Yufeng DONG, Beijing (CN); Youlin QI, Beijing (CN); Yechun WU, Beijing (CN); Yujiao ZHOU, Beijing (CN); Xianggan LI, Beijing (CN); Yingying LI, Beijing (CN)

(21) Appl. No.: 18/263,514

(22) PCT Filed: Jan. 28, 2021
(86) PCT No.: PCT/CN2021/074029

§ 371 Date: Jul. 28, 2023

(51) Int. Cl. C12N 9/10; A01G 22/05; A01G 22/22; A01G 22/25; A01G 22/40; A01G 22/45; A01G 22/50; C12N 15/82

(52) U.S. Cl. CPC C12N 9/1085; C12N 15/8275; C12Y 205/01019; A01G 22/05; A01G 22/22; A01G 22/25; A01G 22/40; A01G 22/45; A01G 22/50

(57) ABSTRACT

The invention discloses gene sequences of a plant EPSPS mutant and its application in plants. As compared to the wild type, the present disclosure describes a plant EPSP synthase (EPSPS) polypeptide which comprises amino acid V mutating to amino acid A corresponding to the location at V403 or the analogous location at V403, wherein the amino acid position corresponds to the amino acid position set forth in SEQ ID NO:1.

### FIELD

The field of the invention relates to the field of molecular biology. More specifically, it pertains to amino acid mutations that confer tolerance to glyphosate and the use of it in the glyphosate tolerate plant.

### BACKGROUND

The 5-enolpyruvylpyruvyl-3-phosphate synthase (EPSPS) encoded by the EPSPS gene is the sixth enzyme in the shikimate pathway. The enzyme catalyzes the conversion of phosphoenolpyruvate (PEP) and 3-phosphoshikimate (S3P) to phosphate and 5-enolpyruvate shikimate-3 phosphate (EPSP). The shikimic acid pathway is widely present in plants, bacteria and fungi, but no such pathway in animals. Glyphosate is one of the most sold chemical herbicides in the world. Its structure is a structural analogue of PEP. By competitively inhibiting the combination of EPSPS and PEP, it results in hindering the synthesis of downstream aromatic amino acids and the imbalance of downstream biosynthesis and metabolism, which effectively kills weeds. Glyphosate herbicide is almost lethal to all plants. In addition to EPSPS in plants, there are also EPSPS in some bacteria that are also sensitive to glyphosate. These glyphosate-sensitive EPSPS are classified as class I, the protein structure of the EPSPS in some microorganisms are similar to the protein structure of the EPSPS in plant, but the amino acid sequence similarity with class I EPSPS sequence is less than 50%, such as the CP4EPSPS, G2-aroA, . . . etc., this kind of EPSPS enzyme is insensitive to glyphosate and is classified as class II.

Glyphosate has become one of the most popular chemical herbicides because of its broad-spectrum, high-efficiency, low-toxicity, and low-cost. Its use in farmland can greatly save the cost of weeding and increase farmers' income. The resistance to glyphosate is one of the most popular traits among the plant varieties created by biotechnology. Up to now, there are about 7 genes in the about 221 approved transgenic events. Only two genes (mEPSPS and 2mEPSPS) are from plants, the other five genes are from microorganisms. As the public is concerned about the safety of introducing foreign genes into genetically modified crops, the product from the chemical mutagenesis and gene editing have become more accepted worldwide. In addition, Gene editing technology has become a new direction for the development of glyphosate-resistant crops because of its high efficiency, site-specific. With only fewer mutation to the gene of the plant, it is possible to obtain the target plant with ideal traits with high commercial value.

The present inventors discovered that certain amino acid mutation of the plant's original EPSPS enzyme can make the plant resistant to glyphosate without affecting the original enzyme function. The inventors also developed the application of these protein mutants and their encoded genes in transgenic plants, gene editing plants or other plant breeding, which can be used to cultivate plants with glyphosate resistance, especially crops, such as agricultural plants.

### SUMMARY

The present disclosure relates to mutation position(s) of the amino acid(s) and the mutation type which comes from the mutated EPSPS. Methods for generating glyphosate tolerant plants are also provided.

In one aspect, the mutated EPSPS polypeptides comprise the mutation V403A, wherein the amino acid mutation position corresponds to the amino acid position set forth in SEQ ID NO:1.

In one aspect, the mutated EPSPS polypeptides comprise the mutation G172A and V403A, wherein the amino acid mutation positions correspond to the amino acid positions set forth in SEQ ID NO:1.

In one aspect, the mutated EPSPS polypeptides comprise the mutation P177S and V403A, wherein the amino acid mutation positions correspond to the amino acid positions set forth in SEQ ID NO:1.

In another aspect, the mutated EPSPS polypeptides comprise the mutation G172A, P177S and/or V403A wherein the amino acid mutation positions correspond to the amino acid positions set forth in SEQ ID NO:1.

In another aspect. the mutated EPSPS polypeptides comprise the mutation T173I, P177S and/or V403A wherein the amino acid mutation positions correspond to the amino acid positions set forth in SEQ ID NO:1.

In another aspect. the mutated EPSPS polypeptides comprise the mutation T173I, P177T and/or V403A wherein the amino acid mutation positions correspond to the amino acid positions set forth in SEQ ID NO:1.

Yet in another aspect, the mutated EPSPS polypeptides comprise the mutation V->A, wherein the position corresponds to the position of the original EPSPS amino acid sequence that is aligned to the position 403 of the SEQ ID NO:1.

Yet in another aspect, the mutated EPSPS polypeptides comprise the mutation G->A and V->A, wherein the positions correspond to the positions of the original EPSPS amino acid sequence that is aligned to the position 172 and 403 of the SEQ ID NO:1.

Yet in another aspect, the mutated EPSPS polypeptides comprise the mutation P->S and V->A, wherein the positions correspond to the positions of the original EPSPS amino acid sequence that is aligned to the position 177 and 403 of the SEQ ID NO:1.

Yet in another aspect, the mutated EPSPS polypeptides comprise the mutation G->A, P->S and/or V->A, wherein the positions correspond to the positions of the original EPSPS amino acid sequence that is aligned to the position 172, 177 and/or 403 of the SEQ ID NO:1.

Yet in another aspect, the mutated EPSPS polypeptides comprise the mutation T->I, P->S and/or V->A, wherein the positions correspond to the positions of the original EPSPS amino acid sequence that is aligned to the position 173, 177 and/or 403 of the SEQ ID NO:1.

Yet in another aspect, the mutated EPSPS polypeptides comprise the mutation T->I, P->T and/or V->A, wherein the positions correspond to the positions of the original EPSPS amino acid sequence that is aligned to the position 173, 177 and/or 403 of the SEQ ID NO:1.

In yet another aspect. the mutated EPSPS polypeptides comprise the mutation G172A, P177S and/or V403A and at least one or more amino acid mutations selected from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO: 1.

In yet another aspect. the mutated EPSPS polypeptides comprise the mutation G172A, P177S and/or V403A and at least one or more analogous amino acid mutations selected from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the analogous amino acid position set forth in SEQ ID NO: 1.

In other embodiments, the mutated EPSPS polypeptide comprises the mutation A70V, G172A, P177S, V403A and/orR514S, wherein each amino acid mutation position corresponds to the amino acid position set forth in SEQ ID NO: 1.

In other embodiments, the mutated EPSPS polypeptide comprises the mutation A73T, G172A, P177S and/or V403A, wherein each amino acid mutation position corresponds to the amino acid position set forth in SEQ ID NO: 1.

In other embodiments, the mutated EPSPS polypeptide comprises the mutation A73T, E86G, G172A, P177S and/or V403A, wherein each amino acid mutation position corresponds to the amino acid position set forth in SEQ ID NO: 1.

In other embodiments, the mutated EPSPS polypeptide comprises the mutation A145G, G172A, P177S, V403A, T442S, and/or D472E, wherein each amino acid mutation position corresponds to the amino acid position set forth in SEQ ID NO: 1.

In other embodiments, the mutated EPSPS polypeptide comprises the mutation G172A, P177S, V403A and/or F502V, wherein each amino acid mutation position corresponds to the amino acid position set forth in SEQ ID NO: 1.

In other embodiments, the mutated EPSPS polypeptide comprises the mutation G172A, P177S, Y252F and/or V403A, wherein each amino acid mutation position corresponds to the amino acid position set forth in SEQ ID NO: 1.

In other embodiments, the mutated EPSPS polypeptide comprises the mutation E137D, G172A, P177S, L253M and/or V403A, wherein each amino acid mutation position corresponds to the amino acid position set forth in SEQ ID NO: 1.

In other embodiments, the mutated EPSPS polypeptide comprises the mutation G172A, P177S, K399N, V403A and/or T466S, wherein each amino acid mutation position corresponds to the amino acid position set forth in SEQ ID NO: 1.

In other embodiments, the mutated EPSPS polypeptide comprises the mutation A73G, L134F, G172A, P177S, G265V, S374R and/or V403A, wherein each amino acid mutation position corresponds to the amino acid position set forth in SEQ ID NO: 1.

In other embodiments, the mutated EPSPS polypeptide comprises the mutation A73K, D114H, V136M, K161E, G172A, P177S, A260T and/or V403A, wherein each amino acid mutation position corresponds to the amino acid position set forth in SEQ ID NO: 1.

In other embodiments, the mutated EPSPS polypeptide comprises the mutation E163K, G172A, P177S, A216V, L253M, H389R and/or V403A, wherein each amino acid mutation position corresponds to the amino acid position set forth in SEQ ID NO: 1.

In other embodiments, the mutated EPSPS polypeptide comprises the mutation G172A, P177S, N246D and/or V403A, wherein each amino acid mutation position corresponds to the amino acid position set forth in SEQ ID NO: 1.

In other embodiments, the mutated EPSPS polypeptide comprises the mutation G172A, M175A, P177S, M258L, K356E, V403A and/or F512I, wherein each amino acid mutation position corresponds to the amino acid position set forth in SEQ ID NO: 1.

In other embodiments, the mutated EPSPS polypeptide comprises the mutation A73S, G172A, P177S, L214F and/or V403A, wherein each amino acid mutation position corresponds to the amino acid position set forth in SEQ ID NO: 1.

In other embodiments, the mutated EPSPS polypeptide comprises the mutation G172A, P177S, V208A, L264H, V403A and/or R514K, wherein each amino acid mutation position corresponds to the amino acid position set forth in SEQ ID NO: 1.

In other embodiments, the mutated EPSPS polypeptide comprises the mutation V113M, G172A, P177S and/or V403A, wherein each amino acid mutation position corresponds to the amino acid position set forth in SEQ ID NO: 1.

Also provided are recombinant DNA constructs comprising the polynucleotides that encode the polypeptide disclosed herein; plant cells comprising a polynucleotide disclosed herein or a recombinant DNA construct comprising such. In some embodiments, the plant cell is a rice cell. In some embodiments, the plant is rice.

In an aspect, methods of generating glyphosate tolerant plants are provided herein. The method comprises expressing in a regenerable plant cell a recombinant DNA construct comprising a polynucleotide operably linked to at least one regulatory sequence, wherein the polynucleotide encodes a plant EPSPS polypeptide that comprises V403A, wherein each amino acid mutation position corresponds to the amino acid position set forth in SEQ ID NO:1 and generating a glyphosate tolerant plant that comprises in its genome the recombinant DNA construct.

In an aspect, methods of generating glyphosate tolerant plants are provided herein. The method comprises expressing in a regenerable plant cell a recombinant DNA construct comprising a polynucleotide operably linked to at least one regulatory sequence, wherein the polynucleotide encodes a plant EPSPS polypeptide that comprises G172A and V403A, wherein each amino acid mutation position corresponds to the amino acid position set forth in SEQ ID NO:1 and generating a glyphosate tolerant plant that comprises in its genome the recombinant DNA construct.

In an aspect, methods of generating glyphosate tolerant plants are provided herein. The method comprises expressing in a regenerable plant cell a recombinant DNA construct comprising a polynucleotide operably linked to at least one regulatory sequence, wherein the polynucleotide encodes a plant EPSPS polypeptide that comprises P177S and V403A, wherein each amino acid mutation position corresponds to the amino acid position set forth in SEQ ID NO:1 and generating a glyphosate tolerant plant that comprises in its genome the recombinant DNA construct.

In some embodiments, the method comprises expressing in a regenerable plant cell a recombinant DNA construct comprising a polynucleotide operably linked to at least one regulatory sequence, wherein the polynucleotide encodes a plant EPSPS polypeptide that comprises G172A, P177S and/or V403A, wherein each amino acid mutation position corresponds to the amino acid position set forth in SEQ ID NO:1 and generating a glyphosate tolerant plant that comprises in its genome the recombinant DNA construct.

In some embodiments, the method comprises expressing in a regenerable plant cell a recombinant DNA construct comprising a polynucleotide operably linked to at least one regulatory sequence, wherein the polynucleotide encodes a plant EPSPS polypeptide that comprises T173I, P177S and/or V403A wherein each amino acid mutation position corresponds to the amino acid position set forth in SEQ ID NO:1 and generating a glyphosate tolerant plant that comprises in its genome the recombinant DNA construct.

In some embodiments, the method comprises expressing in a regenerable plant cell a recombinant DNA construct comprising a polynucleotide operably linked to at least one regulatory sequence, wherein the polynucleotide encodes a plant EPSPS polypeptide that comprises T173I, P177T and/or V403A wherein each amino acid mutation position corresponds to the amino acid position set forth in SEQ ID NO:1 and generating a glyphosate tolerant plant that comprises in its genome the recombinant DNA construct.

In some embodiments, the method comprises expressing in a regenerable plant cell a recombinant DNA construct comprising a polynucleotide operably linked to at least one regulatory sequence, wherein the polynucleotide encodes a plant EPSPS polypeptide that comprises G172A, P177S and/or V403A and at least one amino acid mutation selected from the group consisting of (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO: 1.

In an aspect, methods of generating glyphosate tolerant plants are provided herein. The method comprises expressing in a regenerable plant cell a recombinant DNA construct comprising a polynucleotide operably linked to at least one regulatory sequence, wherein the polynucleotide encodes a plant EPSPS polypeptide that comprises an amino acid mutation that is analogous to V403A, wherein each amino acid mutation position corresponds to the analogous amino acid position set forth in SEQ ID NO:1 and generating a glyphosate tolerant plant that comprises in its genome the recombinant DNA construct.

In an aspect, methods of generating glyphosate tolerant plants are provided herein. The method comprises expressing in a regenerable plant cell a recombinant DNA construct comprising a polynucleotide operably linked to at least one regulatory sequence, wherein the polynucleotide encodes a plant EPSPS polypeptide that comprises an amino acid mutation that is analogous to G172A and an amino acid mutation that is analogous to V403A, wherein each amino acid mutation position corresponds to the analogous amino acid position set forth in SEQ ID NO:1 and generating a glyphosate tolerant plant that comprises in its genome the recombinant DNA construct.

In an aspect, methods of generating glyphosate tolerant plants are provided herein. The method comprises expressing in a regenerable plant cell a recombinant DNA construct comprising a polynucleotide operably linked to at least one regulatory sequence, wherein the polynucleotide encodes a plant EPSPS polypeptide that comprises an amino acid mutation that is analogous to P177S and/or an amino acid mutation that is analogous to V403A, wherein each amino acid mutation position corresponds to the analogous amino acid position set forth in SEQ ID NO:1 and generating a glyphosate tolerant plant that comprises in its genome the recombinant DNA construct.

In some embodiments, the method comprises expressing in a regenerable plant cell a recombinant DNA construct comprising a polynucleotide operably linked to at least one regulatory sequence, wherein the polynucleotide encodes a plant EPSPS polypeptide that comprises an amino acid mutation that is analogous to G172A, an amino acid mutation that is analogous to P177S and/or an amino acid mutation that is analogous to V403A wherein each amino acid mutation position corresponds to the analogous amino acid position set forth in SEQ ID NO:1 and generating a glyphosate tolerant plant that comprises in its genome the recombinant DNA construct.

In some embodiments, the method comprises expressing in a regenerable plant cell a recombinant DNA construct comprising a polynucleotide operably linked to at least one regulatory sequence, wherein the polynucleotide encodes a plant EPSPS polypeptide that comprises an amino acid mutation that is analogous to T173I, an amino acid mutation that is analogous to P177S and/or an amino acid mutation that is analogous to V403A wherein each amino acid mutation position corresponds to the analogous amino acid position set forth in SEQ ID NO:1 and generating a glyphosate tolerant plant that comprises in its genome the recombinant DNA construct.

In some embodiments, the method comprises expressing in a regenerable plant cell a recombinant DNA construct comprising a polynucleotide operably linked to at least one regulatory sequence, wherein the polynucleotide encodes a plant EPSPS polypeptide that comprises an amino acid mutation that is analogous to T173I, an amino acid mutation that is analogous to P177T and/or an amino acid mutation that is analogous to V403A wherein each amino acid mutation position corresponds to the analogous amino acid position set forth in SEQ ID NO:1 and generating a glyphosate tolerant plant that comprises in its genome the recombinant DNA construct.

In some embodiments, the method comprises expressing in a regenerable plant cell a recombinant DNA construct comprising a polynucleotide operably linked to at least one regulatory sequence, wherein the polynucleotide encodes a plant EPSPS polypeptide that comprises an amino acid mutation that is analogous to G172A, an amino acid mutation that is analogous to P177S, and/or an amino acid mutation that is analogous to V403A and at least one amino acid mutation that is analogous to the amino acid mutation selected from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the analogous amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises A70V, G172A, P177S, V403 and/orR514S, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises A73T, G172A, P177S and/or V403A, wherein each amino acid mutation position corresponds to the amino acid position set forth in SEQ ID NO: 1, and generating a glyphosate tolerant plant that comprises in its genome the recombinant DNA construct. In other embodiments, the method comprises expressing in a plant cell a recombinant DNA construct comprising a polynucleotide that encodes a plant EPSPS polypeptide that comprises A73T, E86G, G172A, P177S and/or V403A, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises A145G, G172A, P177S, V403A, T442S, and/or D472E, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises G172A, P177S, V403A and/or F502V, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises G172A, P177S, Y252F and/or V403A, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises E137D, G172A, P177S, L253M and/or V403A, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises G172A, P177S, K399N, V403A and/or T466S, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises A73G, L134G, G172A, P177S, G265V, S374R and/or V403A, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises A73K, D114H, V136M, K161E, G172A, P177S, A260T and/or V403A, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises E163K, G172A, P177S, A216V, L253M, H389R and/or V403A, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises G172A, P177S, N247D and/or V403A, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises G172A, M175A, P177S, M258L, K356E, V403A and/or F512I, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises A73S, G172A, P177S, L214F and/or V403A, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises G172A, P177S, V208A, L264H, V403A and/or R514K, wherein each amino acid mutation position corresponds to the amino acid position set forth in SEQ ID NO: 1, and generating a glyphosate tolerant plant that comprises in its genome the recombinant DNA construct.

In other embodiments, the method comprises expressing in a plant cell a recombinant DNA construct comprising a polynucleotide that encodes a plant EPSPS polypeptide that comprises V113M, G172A, P177S and/or V403A, wherein each amino acid mutation position corresponds to the amino acid position set forth in SEQ ID NO: 1, and generating a glyphosate tolerant plant that comprises in its genome the recombinant DNA construct.

In an aspect, methods of generating glyphosate tolerant plants are provided herein, in which an endogenous plant EPSPS gene (in a plant cell) is modified to encode a glyphosate tolerant EPSPS protein that comprises V403A, wherein the amino acid mutation position corresponds to the amino acid position set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises G172A and V403A wherein the amino acid mutation positions correspond to the amino acid positions set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises P177S and V403A wherein the amino acid mutation positions correspond to the amino acid positions set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises G172A, P177S and/or V403A, wherein the amino acid mutation positions correspond to the amino acid positions set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises T173I, P177S and/or V403A, wherein the amino acid mutation positions correspond to the amino acid positions set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises T173I, P177T and/or V403A, wherein the amino acid mutation positions correspond to the amino acid positions set forth in SEQ ID NO:1; and a glyphosate tolerant plant is grown from the plant cell.

Yet in another aspect, methods of generating glyphosate tolerant plants are provided herein, in which an endogenous plant EPSPS gene (in a plant cell) is modified to encode a glyphosate tolerant EPSPS protein that comprises G172A, P177S and/or V403A, and at least one amino acid selecting from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO:1; and a glyphosate tolerant plant is grown from the plant cell.

In an aspect, methods of generating glyphosate tolerant plants are provided herein, in which an endogenous plant EPSPS gene (in a plant cell) is modified to encode a glyphosate tolerant EPSPS protein that comprises an amino acid mutation that is analogous to V403A, wherein the amino acid mutation position corresponds to the analogous amino acid position set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises an amino acid mutation that is analogous to G172A and an amino acid mutation that is analogous to V403A wherein each amino acid mutation position corresponds to the analogous amino acid position set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises an amino acid mutation that is analogous to P177S and an amino acid mutation that is analogous to V403A wherein each amino acid mutation position corresponds to the analogous amino acid position set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises an amino acid mutation that is analogous to G172A, an amino acid mutation that is analogous to P177S and/or an amino acid mutation that is analogous to V403A wherein each amino acid mutation position corresponds to the analogous amino acid position set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises an amino acid mutation that is analogous to T173I, an amino acid mutation that is analogous to P177S and/or an amino acid mutation that is analogous to V403A wherein each amino acid mutation position corresponds to the analogous amino acid position set forth in SEQ ID NO:1; and a glyphosate tolerant plant is grown from the plant cell.

In another aspect, methods of generating glyphosate tolerant plants are provided herein, in which an endogenous plant EPSPS gene (in a plant cell) is modified to encode a glyphosate tolerant EPSPS protein that comprises an amino acid mutation that is analogous to T173I, an amino acid mutation that is analogous to P177T and/or an amino acid mutation that is analogous to V403A wherein each amino acid mutation position corresponds to the analogous amino acid position set forth in SEQ ID NO:1; and a glyphosate tolerant plant is grown from the plant cell.

Yet in another aspect, methods of generating glyphosate tolerant plants are provided herein, in which an endogenous plant EPSPS gene (in a plant cell) is modified to encode a glyphosate tolerant EPSPS protein that comprises an amino acid mutation that is analogous to G172A, an amino acid mutation that is analogous to P177S, and/or an amino acid mutation that is analogous to V403A and at least one amino acid mutation that is analogous to one amino acid mutation selecting from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the analogous amino acid position set forth in SEQ ID NO:1; and a glyphosate tolerant plant is grown from the plant cell.

In other embodiments, the modified endogenous plant EPSPS gene encodes a glyphosate tolerant EPSPS protein that comprises A70V, G172A, P177S, V403 and/orR514S, wherein each amino acid mutation position corresponds to the amino acid position set forth in SEQ ID NO: 1.

In other embodiments, the modified endogenous plant EPSPS gene encodes a glyphosate tolerant EPSPS protein that comprises A73T, G172A, P177S and/or V403A, wherein each amino acid mutation position corresponds to the amino acid position set forth in SEQ ID NO: 1.

In other embodiments, the modified endogenous plant EPSPS gene encodes a glyphosate tolerant EPSPS protein that comprises A73T, E86G, G172A, P177S and/or V403A, wherein each amino acid mutation position corresponds to the amino acid position set forth in SEQ ID NO: 1.

In other embodiments, the modified endogenous plant EPSPS gene encodes a glyphosate tolerant EPSPS protein that comprises A145G, G172A, P177S, V403A, T442S, and/or D472E, wherein each amino acid mutation position corresponds to the amino acid position set forth in SEQ ID NO: 1.

In other embodiments, the modified endogenous plant EPSPS gene encodes a glyphosate tolerant EPSPS protein that comprises G172A, P177S, V403A and/or F502V, wherein each amino acid mutation position corresponds to the amino acid position set forth in SEQ ID NO: 1.

In other embodiments, the modified endogenous plant EPSPS gene encodes a glyphosate tolerant EPSPS protein that comprises G172A, P177S, Y252F and/or V403A, wherein each amino acid mutation position corresponds to the amino acid position set forth in SEQ ID NO: 1.

In other embodiments, the modified endogenous plant EPSPS gene encodes a glyphosate tolerant EPSPS protein that comprises E137D, G172A, P177S, L253M and/or V403A, wherein each amino acid mutation position corresponds to the amino acid position set forth in SEQ ID NO: 1.

In other embodiments, the modified endogenous plant EPSPS gene encodes a glyphosate tolerant EPSPS protein that comprises G172A, P177S, K399N, V403A and/or T466S, wherein each amino acid mutation position corresponds to the amino acid position set forth in SEQ ID NO: 1.

In other embodiments, the modified endogenous plant EPSPS gene encodes a glyphosate tolerant EPSPS protein that comprises A73G, L134G, G172A, P177S, G265V, S374R and/or V403A, wherein each amino acid mutation position corresponds to the amino acid position set forth in SEQ ID NO: 1.

In other embodiments, the modified endogenous plant EPSPS gene encodes a glyphosate tolerant EPSPS protein that comprises A73K, D114H, V136M, K161E, G172A, P177S, A260T and/or V403A, wherein each amino acid mutation position corresponds to the amino acid position set forth in SEQ ID NO: 1.

In other embodiments, the modified endogenous plant EPSPS gene encodes a glyphosate tolerant EPSPS protein that comprises E163K, G172A, P177S, A216V, L253M, H389R and/or V403A, wherein each amino acid mutation position corresponds to the amino acid position set forth in SEQ ID NO: 1.

In other embodiments, the modified endogenous plant EPSPS gene encodes a glyphosate tolerant EPSPS protein that comprises G172A, P177S, N247D and/or V403A, wherein each amino acid mutation position corresponds to the amino acid position set forth in SEQ ID NO: 1.

In other embodiments, the modified endogenous plant EPSPS gene encodes a glyphosate tolerant EPSPS protein that comprises G172A, M175A, P177S, M258L, K356E, V403A and/or F512I, wherein each amino acid mutation position corresponds to the amino acid position set forth in SEQ ID NO: 1.

In other embodiments, the modified endogenous plant EPSPS gene encodes a glyphosate tolerant EPSPS protein that comprises A73S, G172A, P177S, L214F and/or V403A, wherein each amino acid mutation position corresponds to the amino acid position set forth in SEQ ID NO: 1.

In other embodiments, the modified endogenous plant EPSPS gene encodes a glyphosate tolerant EPSPS protein that comprises G172A, P177S, V208A, L264H, V403A and/or R514K, wherein each amino acid mutation position corresponds to the amino acid position set forth in SEQ ID NO: 1.

In other embodiments, the modified endogenous plant EPSPS gene encodes a glyphosate tolerant EPSPS protein that comprises V113M, G172A, P177S and/or V403A, wherein each amino acid mutation position corresponds to the amino acid position set forth in SEQ ID NO: 1.

The endogenous plant EPSPS gene can be modified by any methods known to one skilled in the art, including, but not limited to: CRISPR/Cas guide RNA-mediated system, Zn-finger nuclease-mediated system, meganulease-mediated system, and/or an oligonucleobase-mediated system.

Polynucleotide constructs that provide a guide RNA in a plant cell are provided herein in which the guide RNA targets an endogenous EPSPS gene of the plant cell and the polynucleotide construct further comprises one or more polynucleotide modification templates to generate a modified endogenous EPSPS gene that encodes a plant EPSPS polypeptide comprising V403A, wherein the amino acid mutation position corresponds to the amino acid set forth in SEQ ID NO:1.

In some embodiments, the polynucleotide construct comprises one or more polynucleotide modification templates to generate a modified endogenous EPSPS gene encoding a plant EPSPS polypeptide that comprises G172A and V403A, wherein the amino acid position corresponds to the amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises P177S and V403A, wherein the amino acid position corresponds to the amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises G172A, P177S and/or V403A, wherein the amino acid position corresponds to the amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises T173I, P177S and/or V403A, wherein the amino acid position corresponds to the amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises T173I, P177T and/or V403A, wherein the amino acid position corresponds to the amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises G172A, P177S, and/or V403A, and at least one or more amino acid mutation selecting from the group consisting of (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid position corresponds to the amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises an amino acid mutation that is analogous to V403A, wherein the amino acid position corresponds to the analogous amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises an amino acid mutation that is analogous to G172A and an amino acid mutation that is analogous to V403A, wherein each amino acid position corresponds to the amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises an amino acid mutation that is analogous to P177S and an amino acid mutation that is analogous to V403A, wherein the amino acid position corresponds to the analogous amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises an amino acid mutation that is analogous to G172A, an amino acid mutation that is analogous to P177S and/or an amino acid mutation that is analogous to V403A, wherein each amino acid position corresponds to the analogous amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises an amino acid mutation that is analogous to T173I, an amino acid mutation that is analogous to P177S and/or an amino acid mutation that is analogous to V403A, wherein each amino acid position corresponds to the analogous amino acid mutation position set forth in SEQ ID NO:1.

In some embodiments, the polynucleotide construct comprises one or more polynucleotide modification templates to generate a modified endogenous EPSPS gene encoding a plant EPSPS polypeptide that comprises an amino acid mutation that is analogous to T173I, an amino acid mutation that is analogous to P177T and/or an amino acid mutation that is analogous to V403A, wherein each amino acid position corresponds to the analogous amino acid mutation position set forth in SEQ ID NO:1.

In other embodiments, the polynucleotide construct comprises one or more polynucleotide modification templates to generate a modified endogenous EPSPS gene encoding a plant EPSPS polypeptide that comprises A70V, G172A, P177S, V403 and/orR514S, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises A73T, G172A, P177S and/or V403A, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises A73T, E86G, G172A, P177S and/or V403A, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises A145G, G172A, P177S, V403A, T442S, and/or D472E, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises G172A, P177S, V403A and/or F502V, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises G172A, P177S, Y252F and/or V403A, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises E137D, G172A, P177S, L253M and/or V403A, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises G172A, P177S, K399N, V403A and/or T466S, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises A73G, L134G, G172A, P177S, G265V, S374R and/or V403A, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises A73K, D114H, V136M, K161E, G172A, P177S, A260T and/or V403A, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises E163K, G172A, P177S, A216V, L253M, H389R and/or V403A, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises G172A, P177S, N247D and/or V403A, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises G172A, M175A, P177S, M258L, K356E, V403A and/or F512I, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises A73S, G172A, P177S, L214F and/or V403A, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises G172A, P177S, V208A, L264H, V403A and/or R514K, wherein each amino acid mutation position corresponds to the amino acid position set forth in SEQ ID NO: 1.

In other embodiments, the polynucleotide construct comprises one or more polynucleotide modification templates to generate a modified endogenous EPSPS gene encoding a plant EPSPS polypeptide that comprises V113M, G172A, P177S and/or V403A, wherein each amino acid mutation position corresponds to the amino acid position set forth in SEQ ID NO: 1.

Also provided herein are glyphosate tolerant plants that express an endogenous EPSPS polypeptide that has V403A, wherein the amino acid position corresponds to the amino acid position set forth in SEQ ID NO:1.

Also provided herein are glyphosate tolerant plants that express an endogenous EPSPS polypeptide that has G172A and V403A, wherein the amino acid position corresponds to the amino acid position set forth in SEQ ID NO:1.

Also provided herein are glyphosate tolerant plants that express an endogenous EPSPS polypeptide that has P177S and V403A, wherein the amino acid position corresponds to the amino acid position set forth in SEQ ID NO:1.

Also provide herein are glyphosate tolerant plants that express an endogenous EPSPS polypeptide that has G172A, P177S and/or V403A, wherein he amino acid position corresponds to the amino acid position set forth in SEQ ID NO:1.

Also provide herein are glyphosate tolerant plants that express an endogenous EPSPS polypeptide that has T173I, P177S and/or V403A, wherein he amino acid position corresponds to the amino acid position set forth in SEQ ID NO:1.

Also provide herein are glyphosate tolerant plants that express an endogenous EPSPS polypeptide that has T173I, P177T and/or V403A, wherein he amino acid position corresponds to the amino acid position set forth in SEQ ID NO:1.

Also provide herein are glyphosate tolerant plants that express an endogenous EPSPS polypeptide that has G172A, P177S and/or V403A, and one or more amino acid mutations selecting from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO:1.

Also provided herein are glyphosate tolerant rice plants that express an endogenous EPSPS polypeptide that has V403A, wherein the amino acid position corresponds to the amino acid position set forth in SEQ ID NO:1. A glyphosate tolerant rice plant may express a plant EPSPS polypeptide having the sequence set forth in SEQ ID NO:3.

Also provided herein are glyphosate tolerant rice plants that express an endogenous EPSPS polypeptide that has G172A and V403A, wherein the amino acid position corresponds to the amino acid position set forth in SEQ ID NO:1. A glyphosate tolerant rice plant may express a plant EPSPS polypeptide having the sequence set forth in SEQ ID NO:4.

Also provided herein are glyphosate tolerant rice plants that express an endogenous EPSPS polypeptide that has P177S and V403A, wherein the amino acid position corresponds to the amino acid position set forth in SEQ ID NO:1. A glyphosate tolerant rice plant may express a plant EPSPS polypeptide having the sequence set forth in SEQ ID NO:5.

Also provided herein are glyphosate tolerant rice plants that express an endogenous EPSPS polypeptide that has G172A, P177S and/or V403A, wherein the amino acid position corresponds to the amino acid position set forth in SEQ ID NO:1. A glyphosate tolerant rice plant may express a plant EPSPS polypeptide having the sequence set forth in SEQ ID NO:6.

Also provided herein are glyphosate tolerant rice plants that express an endogenous EPSPS polypeptide that has T173I, P177S and/or V403A, wherein the amino acid position corresponds to the amino acid position set forth in SEQ ID NO:1. A glyphosate tolerant rice plant may express a plant EPSPS polypeptide having the sequence set forth in SEQ ID NO:37.

Also provided herein are glyphosate tolerant rice plants that express an endogenous EPSPS polypeptide that has T173I, P177T and/or V403A, wherein the amino acid position corresponds to the amino acid position set forth in SEQ ID NO:1. A glyphosate tolerant rice plant may express a plant EPSPS polypeptide having the sequence set forth in SEQ ID NO:38.

Also provide herein are glyphosate tolerant rice plants that express an endogenous EPSPS polypeptide that has G172A, P177S and/or V403A, and one or more amino acid mutations selecting from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO:1.

Also provided herein are glyphosate tolerant rice plants that express an endogenous EPSPS polypeptide that has A70V, G172A, P177S, V403 and/orR514S, wherein each amino acid mutation position corresponds to the amino acid position set forth in SEQ ID NO: 1. A glyphosate tolerant rice plant may express a plant EPSPS polypeptide having the sequence set forth in SEQ ID NO:10.

Also provided herein are glyphosate tolerant rice plants that express an endogenous EPSPS polypeptide that has A73T, G172A, P177S and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:11.

Also provided herein are glyphosate tolerant rice plants that express an endogenous EPSPS polypeptide that has A73T, E86G, G172A, P177S and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:12.

Also provided herein are glyphosate tolerant rice plants that express an endogenous EPSPS polypeptide that has A145G, G172A, P177S, V403A, T442S, and/or D472E, wherein each amino acid mutation position corresponds to the amino acid position set forth in SEQ ID NO: 1. A glyphosate tolerant rice plant may express a plant EPSPS polypeptide having the sequence set forth in SEQ ID NO:13.

Also provided herein are glyphosate tolerant rice plants that express an endogenous EPSPS polypeptide that has G172A, P177S, V403A and/or F502V, wherein each amino acid mutation position corresponds to the amino acid position set forth in SEQ ID NO: 1. A glyphosate tolerant rice plant may express a plant EPSPS polypeptide having the sequence set forth in SEQ ID NO:14.

Also provided herein are glyphosate tolerant rice plants that express an endogenous EPSPS polypeptide that has G172A, P177S, Y252F and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:15.

Also provided herein are glyphosate tolerant rice plants that express an endogenous EPSPS polypeptide that has E137D, G172A, P177S, L253M and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:16.

Also provided herein are glyphosate tolerant rice plants that express an endogenous EPSPS polypeptide that has G172A, P177S, K399N, V403A and/or T466S, wherein each amino acid mutation position corresponds to the amino acid position set forth in SEQ ID NO: 1. A glyphosate tolerant rice plant may express a plant EPSPS polypeptide having the sequence set forth in SEQ ID NO:17.

Also provided herein are glyphosate tolerant rice plants that express an endogenous EPSPS polypeptide that has A73G, L134G, G172A, P177S, G265V, S374R and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:18.

Also provided herein are glyphosate tolerant rice plants that express an endogenous EPSPS polypeptide that has A73K, D114H, V136M, K161E, G172A, P177S, A260T and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:19.

Also provided herein are glyphosate tolerant rice plants that express an endogenous EPSPS polypeptide that has E163K, G172A, P177S, A216V, L253M, H389R and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:20.

Also provided herein are glyphosate tolerant rice plants that express an endogenous EPSPS polypeptide that has G172A, P177S, N247D and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:21.

Also provided herein are glyphosate tolerant rice plants that express an endogenous EPSPS polypeptide that has G172A, M175A, P177S, M258L, K356E, V403A and/or F512I, wherein each amino acid mutation position corresponds to the amino acid position set forth in SEQ ID NO: 1. A glyphosate tolerant rice plant may express a plant EPSPS polypeptide having the sequence set forth in SEQ ID NO:22.

Also provided herein are glyphosate tolerant rice plants that express an endogenous EPSPS polypeptide that has A73S, G172A, P177S, L214F and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:23.

Also provided herein are glyphosate tolerant rice plants that express an endogenous EPSPS polypeptide that has G172A, P177S, V208A, L264H, V403A and/or R514K, wherein each amino acid mutation position corresponds to the amino acid position set forth in SEQ ID NO: 1. A glyphosate tolerant rice plant may express a plant EPSPS polypeptide having the sequence set forth in SEQ ID NO:24.

Also provided herein are glyphosate tolerant rice plants that express an endogenous EPSPS polypeptide that has V113M, G172A, P177S and/or V403A, wherein each amino acid mutation position corresponds to the amino acid position set forth in SEQ ID NO: 1. A glyphosate tolerant rice plant may express a plant EPSPS polypeptide having the sequence set forth in SEQ ID NO:25.

Also provided herein are glyphosate tolerant flax plants that express an endogenous EPSPS polypeptide that has V403A, wherein the amino acid position corresponds to the amino acid position set forth in SEQ ID NO:1. A glyphosate tolerant flax plant may express a plant EPSPS polypeptide having the sequence set forth in SEQ ID NO:27.

Also provided herein are glyphosate tolerant flax plants that express an endogenous EPSPS polypeptide that has T173I and P177T, wherein the amino acid positions correspond to the amino acid positions set forth in SEQ ID NO:1. A glyphosate tolerant flax plant may express a plant EPSPS polypeptide having the sequence set forth in SEQ ID NO:28.

Also provided herein are glyphosate tolerant flax plants that express an endogenous EPSPS polypeptide that has T173I, P177T and/or V403A, wherein each amino acid position corresponds to the amino acid position set forth in SEQ ID NO:1. A glyphosate tolerant flax plant may express a plant EPSPS polypeptide having the sequence set forth in SEQ ID NO:29.

Also provide herein are glyphosate tolerant flax plants that express an endogenous EPSPS polypeptide that has G172A, P177S and/or V403A, wherein each amino acid position corresponds to the amino acid position set forth in SEQ ID NO:1. A glyphosate tolerant flax plant may express a plant EPSPS polypeptide having the sequence set forth in SEQ ID NO:30.

Also provide herein are glyphosate tolerant flax plants that express an endogenous EPSPS polypeptide that has G172A, P177S and/or V403A, and one or more analogous amino acid mutations selecting from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the analogous amino acid position set forth in SEQ ID NO:1.

Also provided herein are glyphosate tolerant wheat plants that express an endogenous EPSPS polypeptide that has V403A, wherein the amino acid position corresponds to the amino acid position set forth in SEQ ID NO: 1. A glyphosate tolerant wheat plant may express a plant EPSPS polypeptide having the sequence set forth in SEQ ID NO:32.

Also provide herein are glyphosate tolerant wheat plants that express an endogenous EPSPS polypeptide that has G172A, P177S, and/or V403A, and one or more analogous amino acid mutations selecting from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the analogous amino acid position set forth in SEQ ID NO:1.

Also provided herein are glyphosate tolerant soybean plants that express an endogenous EPSPS polypeptide that has V403A, wherein the amino acid position corresponds to the amino acid position set forth in SEQ ID NO:1. A glyphosate tolerant soybean plant may express a plant EPSPS polypeptide having the sequence set forth in SEQ ID NO:34.

Also provide herein are glyphosate tolerant soybean plants that express an endogenous EPSPS polypeptide that has G172A, P177S and/or V403A, and one or more analogous amino acid mutations selecting from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the analogous amino acid position set forth in SEQ ID NO:1. Also provided is a method of producing a glyphosate tolerant plant. The method includes introducing into plant cells a recombinant oligonucleotide with a mutant EPSPS gene that expresses a EPSPS protein that is mutated at amino acid position V403 in a rice EPSPS protein (SEQ ID NO: 1) or at an analogous amino acid residue in an EPSPS homolog, wherein V403 is changed to A; selecting a plant cell exhibiting improved tolerance to glyphosate as compared to a corresponding wild-type plant cell; and regenerating a herbicide resistant or tolerant plant having a mutated EPSPS gene from said selected plant cell.

Also provided is a method of producing a glyphosate tolerant plant. The method includes introducing into plant cells a recombinant oligonucleotide with a mutant EPSPS gene that expresses a EPSPS protein that is mutated at amino acid position G172 and V403 in a rice EPSPS protein (SEQ ID NO: 1) or at an analogous amino acid residue in an EPSPS homolog, wherein G172 is changed to A and V403 is changed to A; selecting a plant cell exhibiting improved tolerance to glyphosate as compared to a corresponding wild-type plant cell; and regenerating a herbicide resistant or tolerant plant having a mutated EPSPS gene from said selected plant cell.

Also provided is a method of producing a glyphosate tolerant plant. The method includes introducing into plant cells a recombinant oligonucleotide with a mutant EPSPS gene that expresses a EPSPS protein that is mutated at amino acid position P177 and V403 in a rice EPSPS protein (SEQ ID NO: 1) or at an analogous amino acid residue in an EPSPS homolog, wherein P177 is changed to S and V403 is changed to A; selecting a plant cell exhibiting improved tolerance to glyphosate as compared to a corresponding wild-type plant cell; and regenerating a herbicide resistant or tolerant plant having a mutated EPSPS gene from said selected plant cell.

Also provided is a method of producing a glyphosate tolerant plant. The method includes introducing into plant cells a recombinant oligonucleotide with a mutant EPSPS gene that expresses a EPSPS protein that is mutated at amino acid position G172, P177 and/or V403 in a rice EPSPS protein (SEQ ID NO: 1) or at an analogous amino acid residue in an EPSPS homolog, wherein G172 is changed to A, P177 is changed to S and/or V403 is changed to A; selecting a plant cell exhibiting improved tolerance to glyphosate as compared to a corresponding wild-type plant cell; and regenerating a herbicide resistant or tolerant plant having a mutated EPSPS gene from said selected plant cell.

Also provided is a method of producing a glyphosate tolerant plant. The method includes introducing into plant cells a recombinant oligonucleotide with a mutant EPSPS gene that expresses a EPSPS protein that is mutated at amino acid position T173, P177 and/or V403 in a rice EPSPS protein (SEQ ID NO: 1) or at an analogous amino acid residue in an EPSPS homolog, wherein T173 is changed to I, P177 is changed to S and/or V403 is changed to A; selecting a plant cell exhibiting improved tolerance to glyphosate as compared to a corresponding wild-type plant cell; and regenerating a herbicide resistant or tolerant plant having a mutated EPSPS gene from said selected plant cell.

Also provided is a method of producing a glyphosate tolerant plant. The method includes introducing into plant cells a recombinant oligonucleotide with a mutant EPSPS gene that expresses a EPSPS protein that is mutated at amino acid position T173, P177 and/or V403 in a rice EPSPS protein (SEQ ID NO: 1) or at an analogous amino acid residue in an EPSPS homolog, wherein T173 is changed to I, P177 is changed to T and/or V403 is changed to A; selecting a plant cell exhibiting improved tolerance to glyphosate as compared to a corresponding wild-type plant cell; and regenerating a non-transgenic herbicide resistant or tolerant plant having a mutated EPSPS gene from said selected plant cell.

### BRIEF DESCRIPTION OF THE DRAWINGS

FIG. 1 shows a multiple alignment among SEQ ID NO: 1, SEQ ID NO: 2, SEQ ID NO: 26, SEQ ID NO: 31 and SEQ ID NO: 33.

FIG. 2 shows growth of E. coli cells expressing rice EPSPS protein with G172A, P177S and/or V403A mutants and its combination mutants in different glyphosate concentration media.

FIG. 3 shows growth of E. coli cells expressing rice EPSPS protein with T173I, P177S/T and/or V403A mutants and its combination mutants in different glyphosate concentration media.

FIG. 4 shows the growth status of E. coli cells expressing flax or wheat or soybean EPSPS protein with V403A mutants in different glyphosate concentration media.

FIG. 5 shows the growth status comparison between E. coli cells expressing flax EPSPS protein with G172A, P177S and V403A mutants and E. coli cells expressing CP4 EPSPS protein in different glyphosate concentration media.

FIG. 6 shows the growth status of E. coli cells expressing flax EPSPS protein with G172A, T173I, P177T/S and/or V403A mutants in different glyphosate concentration media.

FIG. 7 shows the glyphosate resistance comparison of E. coli cells expressing rice EPSPS protein with multiple mutants at a concentration of 125 ml glyphosate herbicide.

FIG. 8 shows the construct pKED2 vector.

FIG. 9 shows the pk510 vector which comprises one embodiment of the present disclosure.

FIG. 10 shows comparison between wild-type rice plants and transgenic rice plants and their ability to be glyphosate resistant.

### DETAILED DESCRIPTION

The term “EPSPS homolog” or any variation therefore refers to an EPSPS gene or EPSPS gene product found in another plant species that performs the same or substantially the same biological function as the EPSPS genes disclosed herein and where the nucleic acid sequences or polypeptide sequences (of the EPSPS gene product) are said to be “identical” or at least 50% similar (also referred to as ‘percent identity’ or ‘substantially identical’) as described below. Two polynucleotides or polypeptides are identical if the sequence of nucleotides or amino acid residues, respectively, in the two sequences is the same when aligned for maximum correspondence as described below. The terms “identical” or “percent identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. For polypeptides where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a ‘score of zero, a conservative substitution is given a score between zero and 1. The scoring of conservative substitutions is calculated according to, e.g., the algorithm of Meyers & Miller, Computer Applic. Biol. Sci. 4: 11-17 (1988) e.g., as implemented in the program PC/GENE (Intelligenetics, Mountain View, Calif, USA).

The phrases “substantially identical,” and “percent identity” in the context of two nucleic acids or polypeptides, refer to sequences or subsequences that have at least 50%, advantageously 60%, preferably 70%, more preferably 80%, and most preferably 90-95% nucleotide or amino acid residue identity when aligned for maximum correspondence over a comparison window as measured using one of the following sequence comparison algorithms or by manual alignment and visual inspection. This definition also refers to the complement of a test sequence, which has substantial sequence or subsequence complementarity when the test sequence has substantial identity to a reference sequence.

“Operably linked” refers to a juxtaposition of genetic elements, wherein the elements are in a relationship permitting them to operate in the expected manner. For instance, a 5′ regulatory sequence is operably linked to a coding sequence if the 5′ regulatory sequence functions to initiate transcription of the coding sequence.

As used herein, a “glyphosate-resistant” cell or plant refers to a cell or plant that can survive or continue to grow in the presence of certain concentrations of glyphosate that typically kill or inhibit the growth of other cells or plants. Growth includes, for instance, photosynthesis, increased rooting, increased height, increased mass, or development of new leaves. In one embodiment, a glyphosate-resistant cell can grow and divide on a culture medium containing 50 mg/l or more glyphosate. Preferably, a glyphosate-resistant cell can grow and divide on a culture medium containing 100 mg/l or more glyphosate, such as 200 mg/1, 300 mg/l or 400 mg/l glyphosate. More preferably, a glyphosate-resistant cell can grow and divide on a culture medium containing 500 mg/l or more glyphosate, such as 600 mg/1. For purposes of the present invention, the term “glyphosate” includes any herbicidally effective form of N-phosphonomethylglycine (including any salt thereof) and other forms which result in the production of the glyphosate anion in plants.

### Sequence Listing Details

SEQ ID NO:1 is the full length amino acid sequence of a wild type rice EPSPS protein (GenBank entry XP_015643046.1), wherein its N-terminal comprises a chloroplasttransit peptide. SEQ ID NO:1 is to be used herein as a reference EPSPS sequence and all mutation positionsare marked according to SEQ ID NO:1.

SEQ ID NO:2 is the amino acid sequence of a wild type rice EPSPS protein. Comparing to SEQ ID NO: 1, the chlorplast transit peptide is removed from SEQ ID NO:2.

SEQ ID NO:3 is the mutated version of the EPSPS sequence from rice that contains V403A mutations.

SEQ ID NO:4 is the mutated version of the EPSPS sequence from rice that contains G172A and V403A mutations.

SEQ ID NO:5 is the mutated version of the EPSPS sequence from rice that contains P177S and V403A mutations.

SEQ ID NO:6 is the mutated version of the EPSPS sequence from rice that contains G172A, P177S and V403A mutations.

SEQ ID NO:7 is the mutated version of the EPSPS sequence from rice that contains G172A mutations.

SEQ ID NO:8 is the mutated version of the EPSPS sequence from rice that contains P177S mutations.

SEQ ID NO:9 is the mutated version of the EPSPS sequence from rice that contains G172A and P177S mutations.

SEQ ID NO:10 is the mutated version of the EPSPS sequence from rice that contains A70V, G172A, P177S, V403A and R514S mutations.

SEQ ID NO:11 is the mutated version of the EPSPS sequence from rice that contains A73T, G172A, P177S and V403A mutations.

SEQ ID NO:12 is the mutated version of the EPSPS sequence from rice that contains A73T, E86G, G172A, P177S and V403A mutations.

SEQ ID NO:13 is the mutated version of the EPSPS sequence from rice that contains A145G, G172A, P177S, V403A, T442S and D472E mutations.

SEQ ID NO:14 is the mutated version of the EPSPS sequence from rice that contains G172A, P177S, V403A and F502V mutations.

SEQ ID NO:15 is the mutated version of the EPSPS sequence from rice that contains G172A, P177S, Y252F and V403A mutations.

SEQ ID NO:16 is the mutated version of the EPSPS sequence from rice that contains E137D, G172A, P177S, L253M and V403A mutations.

SEQ ID NO:17 is the mutated version of the EPSPS sequence from rice that contains G172A, P177S, K399N, V403A and T466S mutations.

SEQ ID NO:18 is the mutated version of the EPSPS sequence from rice that contains A73G, L134F, G172A, P177S, G265V, S374R and V403A mutations.

SEQ ID NO:19 is the mutated version of the EPSPS sequence from rice that contains A73K, D114H, V136M, K161E, G172A, P177S, A260T and V403A mutations.

SEQ ID NO:20 is the mutated version of the EPSPS sequence from rice that contains E163K, G172A, P177S, A216V, L253M, H389R and V403A mutations.

SEQ ID NO:21 is the mutated version of the EPSPS sequence from rice that contains G172A, P177S, N246D and V403A mutations.

SEQ ID NO:22 is the mutated version of the EPSPS sequence from rice that contains G172A, M175A, P177S, M258L, K356E, V403A and F512I mutations.

SEQ ID NO:23 is the mutated version of the EPSPS sequence from rice that contains A73S, G172A, P177S, L214F and V403A mutations.

SEQ ID NO:24 is the mutated version of the EPSPS sequence from rice that contains G172A, P177S, V208A, L264H, V403A and R514K mutations.

SEQ ID NO:25 is the mutated version of the EPSPS sequence from rice that contains V113M, G172A, P177S and V403A mutations.

SEQ ID NO:26 is the amino acid sequence of a wild type flax EPSPS2 translated from Lus10000788 (Sauer N J, Narváez-Vásquez, Javier, Mozoruk J, et al. Oligonucleotide-Mediated Genome Editing Provides Precision and Function to Engineered Nucleases and Antibiotics in Plants[J]. Plant Physiology, 2016:1917-1928.), wherein its chloroplast transit peptide is removed from SEQ ID NO:26.

SEQ ID NO:27 is the mutated version of the EPSPS sequence from flax that contains V403A mutations.

SEQ ID NO:28 is the mutated version of the EPSPS sequence from flax that contains T173I and P177T mutations.

SEQ ID NO:29 is the mutated version of the EPSPS sequence from flax that contains T173I, P177T and V403A mutations.

SEQ ID NO:30 is the mutated version of the EPSPS sequence from flax that contains G172A, P177S and V403A mutations.

SEQ ID NO:31 is the amino acid sequence of a wild type wheat EPSPS protein presented as GenBank entry ALK27163.1, wherein its chloroplast transit peptide is removed from SEQ ID NO:31.

SEQ ID NO:32 is the mutated version of the EPSPS sequence from wheat that contains V403A mutations.

SEQ ID NO:33 is the amino acid sequence of a wild type soybean EPSPS protein presented as GenBank entry XP_003521857.1, wherein its chloroplast transit peptide is removed from SEQ ID NO:33.

SEQ ID NO:34 is the mutated version of the EPSPS sequence from soybean that contains V403A mutations.

SEQ ID NO:35 is the mutated version of the EPSPS sequence from rice that contains T173I and P177S mutations.

SEQ ID NO:36 is the mutated version of the EPSPS sequence from rice that contains T173I and P177T mutations.

SEQ ID NO:37 is the mutated version of the EPSPS sequence from rice that contains T173I, P177S and V403A mutations.

SEQ ID NO:38 is the mutated version of the EPSPS sequence from rice that contains T173I, P177T and V403A mutations.

SEQ ID NO:39 is the amino acid sequence of wild type EPSPS from CP4 (Agrobacterium tumefaciens).

### 1. Compositions

### A. EPSPS Polypeptides

Various methods and compositions are provided which employ mutated polypeptides having EPSPS activity. Such EPSPS polypeptides comprise V403A mutation, wherein the amino acid position corresponds to the amino acid position set forth in SEQ ID NO:1. Such EPSPS polypeptides comprise G172A and V403A mutation, wherein the amino acid positions correspond to the amino acid positions set forth in SEQ ID NO:1. Such EPSPS polypeptides comprise P177S and V403A mutation, wherein the amino acid positions correspond to the amino acid positions set forth in SEQ ID NO:1.

In some embodiments, the EPSPS polypeptides comprise G172A, P177S and/or V403A, wherein each amino acid position corresponds to the amino acid position set forth in SEQ ID NO: 1.

In some embodiments, the EPSPS polypeptides comprise T173I, P177S and/or V403A, wherein each amino acid position corresponds to the amino acid position set forth in SEQ ID NO: 1.

In some embodiments, the EPSPS polypeptides comprise T173I, P177T and/or V403A, wherein each amino acid position corresponds to the amino acid position set forth in SEQ ID NO: 1.

In other embodiments, the EPSPS polypeptides comprise G172A, P177S, V403A and at least one or more amino acid mutations selected from the group consisting of (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO:1.

In other embodiments, a plant EPSPS polypeptide comprises A70V, G172A, P177S, V403 and/or R514S mutations. In still other embodiments, a plant EPSPS polypeptide comprises A73T, G172A, P177S and/or V403A mutations. In still other embodiments, a plant EPSPS polypeptide comprises A73T, E86G, G172A, P177S and/or V403A mutations. In still other embodiments, a plant EPSPS polypeptide comprises A145G, G172A, P177S, V403A, T442S and/or D472E mutations. In still other embodiments, a plant EPSPS polypeptide comprises G172A, P177S, V403A and/or F502V mutations. In still other embodiments, a plant EPSPS polypeptide comprises G172A, P177S, Y252F and/or V403A mutations. In still other embodiments, a plant EPSPS polypeptide comprises E137D, G172A, P177S, L253M and/or V403A mutations. In still other embodiments, a plant EPSPS polypeptide comprises G172A, P177S, K399N, V403A and/or T466S mutations. In still other embodiments, a plant EPSPS polypeptide comprises A73G, L134F, G172A, P177S, G265V, S374R and/or V403A mutations. In still other embodiments, a plant EPSPS polypeptide comprises A73K, D114H, V136M, K161E, G172A, P177S, A260T and/or V403A mutations. In still other embodiments, a plant EPSPS polypeptide comprises E163K, G172A, P177S, A216V, L253M, H389R and/or V403A mutations. In still other embodiments, a plant EPSPS polypeptide comprises G172A, P177S, N246D and/or V403A mutations. In still other embodiments, a plant EPSPS polypeptide comprises G172A, M175A, P177S, M258L, K356E, V403A and/or F512I mutations. In still other embodiments, a plant EPSPS polypeptide comprises A73S, G172A, P177S, L214F and/or V403A mutations. In still other embodiments, a plant EPSPS polypeptide comprises G172A, P177S, V208A, L264H, V403A and/or R514K mutations. In still other embodiments, a plant EPSPS polypeptide comprises V113M, G172A, P177S and/or V403A mutations.

The EPSPS polypeptides mutants disclosed herein may have improved catalytic capacity in the presence of glyphosate when compared to wild type EPSPS polypeptides.

As disclosed herein, the EPSPS mutant comprising V403A can improve variety of plants resistant to glyphosate while still maintaining its own biological enzyme catalytic activity. However, the location of this amino acid V403 in the EPSPS may vary within other different plant species. The analogous mutation V->A on the analogous location may also improve other plants resistant to glyphosate. The mutation location can be decided in two steps. First step is to align the wild type plant EPSPS to rice EPSPS sequence (SEQ ID NO:1). Second step is to find the aligned position corresponding to the position 403 on the rice EPSPS sequence.

Previous study also discloses that G172 and P177 are also two conserved position on most of the EPSPS sequence. However, the location of this amino acid G172 or P177 in the EPSPS sequence may vary with some other species. The analogous mutation G->A, P->S and/or V->A on the analogous location may also improve some other plants resistant to glyphosate. The mutation location can be decided in two steps. First step is to align the wild type plant EPSPS to rice EPSPS sequence. Second step is to find the aligned position corresponding to the position 172, 177 and/or 403 on the rice EPSPS sequence.

Previous study also discloses that T173 and P177 are also two conserved position on most of the EPSPS sequence. However, the location of this amino acid T173 or P177 in the EPSPS sequence may vary with some other species. The analogous mutation T->I, P->S/T and/or V->A on the analogous location may also improve some other plants resistant to glyphosate. The mutation location can be decided in two steps. First step is to align the wild type plant EPSPS to rice EPSPS sequence. Second step is to find the aligned position corresponding to the position 173, 177 and/or 403 on the rice EPSPS sequence.

Various methods and compositions are provided which employ mutated polypeptides having EPSPS activity. Such EPSPS polypeptides comprise an amino acid mutation that is analogous to V403A mutation, wherein the amino acid position corresponds to the analogous amino acid position set forth in SEQ ID NO:1. Such EPSPS polypeptides comprise an amino acid mutation that is analogous to G172A and an amino acid mutation that is analogous to V403A mutation, wherein each amino acid position corresponds to the analogous amino acid position set forth in SEQ ID NO:1. Such EPSPS polypeptides comprise an amino acid mutation that is analogous to P177S and an amino acid mutation that is analogous to V403A mutation, wherein each amino acid position corresponds to the analogous amino acid position set forth in SEQ ID NO:1.

In some embodiments, the EPSPS polypeptides comprise an amino acid mutation that is analogous to G172A, an amino acid mutation that is analogous to P177S and an amino acid mutation that is analogous to V403A, wherein each amino acid position corresponds to the analogous amino acid position set forth in SEQ ID NO: 1.

In some embodiments, the EPSPS polypeptides comprise an amino acid mutation that is analogous to T173I, an amino acid mutation that is analogous to P177S and/or an amino acid mutation that is analogous to V403A, wherein each amino acid position corresponds to the analogous amino acid position set forth in SEQ ID NO: 1.

In some embodiments, the EPSPS polypeptides comprise an amino acid mutation that is analogous to T173I, an amino acid mutation that is analogous to P177T and/or an amino acid mutation that is analogous to V403A, wherein each amino acid position corresponds to the analogous amino acid position set forth in SEQ ID NO: 1.

In other embodiments, the EPSPS polypeptides comprise an amino acid mutation that is analogous to G172A, an amino acid mutation that is analogous to P177S, an amino acid mutation that is analogous to V403A and at least one or more analogous amino acid mutations selected from the group consisting of (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the analogous amino acid position set forth in SEQ ID NO:1.

### B. Plants and Other Host Cells of Interest

Further provided are engineered host cells that are transduced (transformed or transfected) with one or more EPSPS sequences or active variants or fragments thereof. The EPSPS polypeptides can be expressed in any organism, including non-animal cells such as plants, yeast, fungi, bacteria and the like.

Plants, plant cells, plant parts and seeds, and grain having the EPSPS sequences disclosed herein are also provided. In specific embodiments, the plants and/or plant parts have stably incorporated at least one heterologous EPSPS polypeptide disclosed herein or an active variant or fragment thereof. In addition, the plants or organism of interest can comprise multiple EPSPS polynucleotides.

In specific embodiments, the heterologous plant EPSPS polynucleotide in the plant or plant part is operably linked to a heterologous regulatory element, such as but not limited to a constitutive, tissue-preferred, or other promoter for expression in plants or a constitutive enhancer.

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cos, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

The EPSPS sequences and active variants and fragments thereof disclosed herein may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, barley (Hordeum vulgare), oat (Avena sativa), alfalfa (Medicago sativa), rice (Oryza sativa), rye (Secale cereale), sorghum (Sorghum bicolor, Sorghum vulgare), sweet sorghum (Sorghum dochna) millet (e.g., pearl millet (Pennisetum glaucum), proso millet (Panicum miliaceum), foxtail millet (Setaria italica), finger millet (Eleusine coracana)), hemp (Cannabis sativa L.), kentucky bluegrass (Poa pratensis), tall fescue (Festuca arundinacea), creeping bentgrass (Agrostis stolonfera), sunflower (Helianthus annuus), safflower (Carthamus tinctorius), wheat (Triticum aestivum), soybean (Glycine max), flax (Linum usitatissimum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), tomato (Solanum lycopersicum), potato (Solanum tuberosum), apple tree (Malus pumila), grapevine (Vitis vinifera L.), cucumber (C. sativus), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), vegetables, ornamentals, conifers, turf grasses (including cool seasonal grasses and warm seasonal grasses).

Vegetables include tomatoes (Lycopersicon esculentum), lettuce (e.g., Lactuca sativa), green beans (Phaseolus vulgaris), lima beans (Phaseoluslimensis), peas (Lathyrus spp.), potato (Solanum tuberosum), carrot (Daucus carota subsp. Sativus), onion (Allium cepa) and members of the genus Cucumis such as cucumber (C. sativus), cantaloupe (C. cantalupensis), and musk melon (C. melo). Ornamentals include azalea (Rhododendron spp.), hydrangea (Macrophyllahydrangea), hibiscus (Hibiscus rosasanensis), roses (Rosa spp.), tulips (Tulipa spp.), daffodils (Narcissus spp.), petunias (Petunia hybrida), carnation (Dianthuscaryophyllus), poinsettia (Euphorbia pulcherrima), and chrysanthemum.

Other plants of interest include Poaceae species such as Eleusine Indica, Aegliops tauschil subse. Tausch, Setatia Italica, Sorghum Halepense, Triticum Aestivum, Lolium Multiflorum, Oryza Meyeriana var. granulata, Brachypodium Distachyon, Bromus Tectorum, Lolium Rigidum, Panicum Miliaceum, Dichanthelium Oligosanthes, Hordeum Vulgare, Panicum Hali etc. Other plants of interest include Asteraceae species such as Erigeron Canadensis, Artemeisia Annua, Tanacetum Cinerariifolium etc. Other plants of interest include Arecaceae species such as Elaeis Guineensis, Phoenix Dactylifera etc. Other plants of interest include Orchidaceae such as Apostasia Shenzhenica, Dendrobium Catenatum, Phalaenopsis etc. Other plants of interest include Rosaceae such as Fragaria Ananassa, Rosa Chinensisetc.

Other plants of interest include grain plants that provide seeds of interest, oil seed plants, and Ieguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Additional host cells of interest can be a eukaryotic cell, a protoplast, a tissue culture cell, prokaryotic cell, and/or a bacterial cell, such as E. coli and others.

For example, in some embodiments, glyphosate tolerant rice plants are provided, in which the glyphosate tolerant rice plants express an endogenous EPSPS polypeptide that comprises V403A, wherein the amino acid position corresponds to the amino acid position set forth in SEQ ID NO:1. Further, the glyphosate tolerant rice plant may express an endogenous EPSPS polypeptide that comprises G172A, P177S and/or V403A, wherein each amino acid position corresponds to the amino acid position set forth in SEQ ID NO: 1. Further, the glyphosate tolerant rice plant may express an endogenous EPSPS polypeptide that comprises T173I, P177S and/or V403A, wherein the amino acid position corresponds to the amino acid position set forth in SEQ ID NO: 1. Further, the glyphosate tolerant rice plant may express an endogenous EPSPS polypeptide that comprises T173I, P177T and/or V403A, wherein each amino acid position corresponds to the amino acid position set forth in SEQ ID NO: 1. Further, the glyphosate tolerant rice plant may express an endogenous EPSPS polypeptide that comprises G172A, P177S, and/or V403A, and at least one amino acid mutations selected from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO:1.

Further, the glyphosate tolerant rice plant may express an endogenous EPSPS polypeptide that comprises A70V, G172A, P177S, V403 and/or R514S mutation. Further, the glyphosate tolerant rice plant may express an endogenous EPSPS polypeptide that comprises A73T, G172A, P177S and/or V403A mutation. Further, the glyphosate tolerant rice plant may express an endogenous EPSPS polypeptide that comprises A73T, E86G, G172A, P177S and/or V403A mutation. Further, the glyphosate tolerant rice plant may express an endogenous EPSPS polypeptide that comprises A145G, G172A, P177S, V403A, T442S and/or D472E mutations. Further, the glyphosate tolerant rice plant may express an endogenous EPSPS polypeptide that comprises G172A, P177S, V403A and/or F502V mutations. Further, the glyphosate tolerant rice plant can express an endogenous EPSPS polypeptide that comprises G172A, P177S, Y252F and/or V403A mutations. Further, the glyphosate tolerant rice plant may express an endogenous EPSPS polypeptide that comprises E137D, G172A, P177S, L253M and/or V403A mutations. Further, the glyphosate tolerant rice plant may express an endogenous EPSPS polypeptide that comprises G172A, P177S, K399N, V403A and/or T466S mutations. Further, the glyphosate tolerant rice plant may express an endogenous EPSPS polypeptide that comprises A73G, L134F, G172A, P177S, G265V, S374R and/or V403A mutations. Further, the glyphosate tolerant rice plant may express an endogenous EPSPS polypeptide that comprises A73K, D114H, V136M, K161E, G172A, P177S, A260T and/or V403A mutations. Further, the glyphosate tolerant rice plant may express an endogenous EPSPS polypeptide that comprises E163K, G172A, P177S, A216V, L253M, H389R and/or V403A mutations. Further, the glyphosate tolerant rice plant may express an endogenous EPSPS polypeptide that comprises G172A, P177S, N246D and/or V403A mutations. Further, the glyphosate tolerant rice plant may express an endogenous EPSPS polypeptide that comprises G172A, M175A, P177S, M258L, K356E, V403A and/or F512I mutations. Further, the glyphosate tolerant rice plant may express an endogenous EPSPS polypeptide that comprises A73S, G172A, P177S, L214F and/or V403A mutations. Further, the glyphosate tolerant rice plant may express an endogenous EPSPS polypeptide that comprises G172A, P177S, V208A, L264H, V403A and/or R514K mutations. Further, the glyphosate tolerant rice plant may express an endogenous EPSPS polypeptide that comprises V113M, G172A, P177S and/or V403A mutations.

For example, in some embodiments, glyphosate tolerant flax plants are provided, in which the glyphosate tolerant flax plants express an endogenous EPSPS polypeptide that comprises V403A, wherein the amino acid position corresponds to the amino acid position set forth in SEQ ID NO:1. Further, the glyphosate tolerant flax plant may express an endogenous EPSPS polypeptide that comprises G172A, P177S and/or V403A, wherein the amino acid position corresponds to the amino acid position set forth in SEQ ID NO: 1. Further, the glyphosate tolerant flax plant may express an endogenous EPSPS polypeptide that comprises G172A, P177S, and/or V403A and at least one amino acid mutations selected from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO:1.

Further, the glyphosate tolerant flax plant may express an endogenous EPSPS polypeptide that comprises A70V, G172A, P177S, V403 and/or R514S mutation. Further, the glyphosate tolerant flax plant may express an endogenous EPSPS polypeptide that comprises A73T, G172A, P177S and/or V403A mutation. Further, the glyphosate tolerant flax plant may express an endogenous EPSPS polypeptide that comprises A73T, E86G, G172A, P177S and/or V403A mutation. Further, the glyphosate tolerant flax plant may express an endogenous EPSPS polypeptide that comprises A145G, G172A, P177S, V403A, T442S and/or D472E mutations. Further, the glyphosate tolerant flax plant may express an endogenous EPSPS polypeptide that comprises G172A, P177S, V403A and/or F502V mutations. Further, the glyphosate tolerant flax plant may express an endogenous EPSPS polypeptide that comprises G172A, P177S, Y252F and/or V403A mutations. Further, the glyphosate tolerant flax plant may express an endogenous EPSPS polypeptide that comprises E137D, G172A, P177S, L253M and/or V403A mutations. Further, the glyphosate tolerant flax plant may express an endogenous EPSPS polypeptide that comprises G172A, P177S, K399N, V403A and/or T466S mutations. Further, the glyphosate tolerant flax plant may express an endogenous EPSPS polypeptide that comprises A73G, L134F, G172A, P177S, G265V, S374R and/or V403A mutations. Further, the glyphosate tolerant flax plant may express an endogenous EPSPS polypeptide that comprises A73K, D114H, V136M, K161E, G172A, P177S, A260T and/or V403A mutations. Further, the glyphosate tolerant flax plant may express an endogenous EPSPS polypeptide that comprises E163K, G172A, P177S, A216V, L253M, H389R and/or V403A mutations. Further, the glyphosate tolerant flax plant may express an endogenous EPSPS polypeptide that comprises G172A, P177S, N246D and/or V403A mutations. Further, the glyphosate tolerant flax plant may express an endogenous EPSPS polypeptide that comprises G172A, M175A, P177S, M258L, K356E, V403A and/or F512I mutations. Further, the glyphosate tolerant flax plant may express an endogenous EPSPS polypeptide that comprises A73S, G172A, P177S, L214F and/or V403A mutations. Further, the glyphosate tolerant flax plant may express an endogenous EPSPS polypeptide that comprises G172A, P177S, V208A, L264H, V403A and/or R514K mutations. Further, the glyphosate tolerant flax plant may express an endogenous EPSPS polypeptide that comprises V113M, G172A, P177S and/or V403A mutations.

For example, in some embodiments, glyphosate tolerant wheat plants are provided, in which the glyphosate tolerant wheat plants express an endogenous EPSPS polypeptide that comprises V403A, wherein the amino acid position corresponds to the amino acid position set forth in SEQ ID NO:1. Further, the glyphosate tolerant wheat plant may express an endogenous EPSPS polypeptide that comprises G172A, P177S and/or V403A, wherein the amino acid position corresponds to the amino acid position set forth in SEQ ID NO: 1. Further, the glyphosate tolerant wheat plant may express an endogenous EPSPS polypeptide that comprises G172A, P177S, and/or V403A, and at least one amino acid mutations selected from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO:1.

Further, the glyphosate tolerant wheat plant may express an endogenous EPSPS polypeptide that comprises A70V, G172A, P177S, V403A and/or R514S mutation. Further, the glyphosate tolerant wheat plant may express an endogenous EPSPS polypeptide that comprises A73T, G172A, P177S and/or V403A mutation. Further, the glyphosate tolerant wheat plant may express an endogenous EPSPS polypeptide that comprises A73T, E86G, G172A, P177S and/or V403A mutation. Further, the glyphosate tolerant wheat plant may express an endogenous EPSPS polypeptide that comprises A145G, G172A, P177S, V403A, T442S and/or D472E mutations. Further, the glyphosate tolerant wheat plant may express an endogenous EPSPS polypeptide that comprises G172A, P177S, V403A and/or F502V mutations. Further, the glyphosate tolerant wheat plant may express an endogenous EPSPS polypeptide that comprises G172A, P177S, Y252F and/or V403A mutations. Further, the glyphosate tolerant wheat plant may express an endogenous EPSPS polypeptide that comprises E137D, G172A, P177S, L253M and/or V403A mutations. Further, the glyphosate tolerant wheat plant may express an endogenous EPSPS polypeptide that comprises G172A, P177S, K399N, V403A and/or T466S mutations. Further, the glyphosate tolerant wheat plant may express an endogenous EPSPS polypeptide that comprises A73G, L134F, G172A, P177S, G265V, S374R and/or V403A mutations. Further, the glyphosate tolerant wheat plant may express an endogenous EPSPS polypeptide that comprises A73K, D114H, V136M, K161E, G172A, P177S, A260T and/or V403A mutations. Further, the glyphosate tolerant wheat plant may express an endogenous EPSPS polypeptide that comprises E163K, G172A, P177S, A216V, L253M, H389R and/or V403A mutations. Further, the glyphosate tolerant wheat plant may express an endogenous EPSPS polypeptide that comprises G172A, P177S, N246D and/or V403A mutations. Further, the glyphosate tolerant wheat plant may express an endogenous EPSPS polypeptide that comprises G172A, M175A, P177S, M258L, K356E, V403A and/or F512I mutations. Further, the glyphosate tolerant wheat plant may express an endogenous EPSPS polypeptide that comprises A73S, G172A, P177S, L214F and/or V403A mutations. Further, the glyphosate tolerant wheat plant may express an endogenous EPSPS polypeptide that comprises G172A, P177S, V208A, L264H, V403A and/or R514K mutations. Further, the glyphosate tolerant wheat plant may express an endogenous EPSPS polypeptide that comprises V113M, G172A, P177S and/or V403A mutations.

For example, in some embodiments, glyphosate tolerant soybean plants are provided, in which the glyphosate tolerant soybean plants express an endogenous EPSPS polypeptide that comprises V403A, wherein the amino acid position corresponds to the amino acid position set forth in SEQ ID NO:1. Further, the glyphosate tolerant soybean plant may express an endogenous EPSPS polypeptide that comprises G172A, P177S and/or V403A, wherein the amino acid position corresponds to the amino acid position set forth in SEQ ID NO: 1. Further, the glyphosate tolerant soybean plant may express an endogenous EPSPS polypeptide that comprises G172A, P177S, and/or V403A, and at least one amino acid mutations selected from the group consisting of: (1) A70V, (2) A73T/V/S/K/G, (3) VI13M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO:1.

Further, the glyphosate tolerant soybean plant may express an endogenous EPSPS polypeptide that comprises A70V, G172A, P177S, V403A and/or R514S mutation. Further, the glyphosate tolerant soybean plant may express an endogenous EPSPS polypeptide that comprises A73T, G172A, P177S and/or V403A mutation. Further, the glyphosate tolerant soybean plant may express an endogenous EPSPS polypeptide that comprises A73T, E86G, G172A, P177S and/or V403A mutation. Further, the glyphosate tolerant soybean plant may express an endogenous EPSPS polypeptide that comprises A145G, G172A, P177S, V403A, T442S and/or D472E mutations. Further, the glyphosate tolerant soybean plant may express an endogenous EPSPS polypeptide that comprises G172A, P177S, V403A and/or F502V mutations. Further, the glyphosate tolerant soybean plant may express an endogenous EPSPS polypeptide that comprises G172A, P177S, Y252F and/or V403A mutations. Further, the glyphosate tolerant soybean plant may express an endogenous EPSPS polypeptide that comprises E137D, G172A, P177S, L253M and/or V403A mutations. Further, the glyphosate tolerant soybean plant may express an endogenous EPSPS polypeptide that comprises G172A, P177S, K399N, V403A and/or T466S mutations. Further, the glyphosate tolerant soybean plant may express an endogenous EPSPS polypeptide that comprises A73G, L134F, G172A, P177S, G265V, S374R and/or V403A mutations. Further, the glyphosate tolerant soybean plant may express an endogenous EPSPS polypeptide that comprises A73K, D114H, V136M, K161E, G172A, P177S, A260T and/or V403A mutations. Further, the glyphosate tolerant soybean plant may express an endogenous EPSPS polypeptide that comprises E163K, G172A, P177S, A216V, L253M, H389R and/or V403A mutations. Further, the glyphosate tolerant soybean plant may express an endogenous EPSPS polypeptide that comprises G172A, P177S, N246D and/or V403A mutations. Further, the glyphosate tolerant soybean plant may express an endogenous EPSPS polypeptide that comprises G172A, M175A, P177S, M258L, K356E, V403A and/or F512I mutations. Further, the glyphosate tolerant soybean plant may express an endogenous EPSPS polypeptide that comprises A73S, G172A, P177S, L214F and/or V403A mutations. Further, the glyphosate tolerant soybean plant may express an endogenous EPSPS polypeptide that comprises G172A, P177S, V208A, L264H, V403A and/or R514K mutations. Further, the glyphosate tolerant soybean plant may express an endogenous EPSPS polypeptide that comprises V113M, G172A, P177S and/or V403A mutations.

### C. Stacking Other Traits of Interest

In one embodiment, the present invention relates to transgenic plants having the polynucleotide construct having a promotor operably linked to a mutated EPSPS as described above. In addition, other herbicide tolerant proteins may be inserted in the transgenic plants to produce multiple herbicide resistant plants.

As used herein, the term “stacked” includes having the multiple traits present in the same plant or organism of interest. A trait, as used herein, refers to the phenotype derived from a particular sequence or groups of sequences. In one embodiment, the molecular stack comprises at least one additional polynucleotide that also confers tolerance to at least one sequence that confers tolerance to glyphosate by the same and/or different mechanism and/or at least one additional polynucleotide that confers tolerance to a second herbicide.

The mechanism of glyphosate tolerance produced by the EPSPS sequences disclosed herein may be combined with other modes of herbicide resistance to provide host cells, plants, plant explants and plant cells that are tolerant to glyphosate and one or more other herbicides. For instance, the mechanism of glyphosate tolerance conferred by EPSPS may be combined with other modes of glyphosate tolerance known in the art. In other embodiments, the plant or plant cell or plant part having the EPSPS sequence or an active variant or fragment thereof may be stacked with, for example, one or more sequences that confer tolerance to: an ALS inhibitor; an HPPD inhibitor; 2,4-D; Other phenoxy auxin herbicides; aryloxyphenoxypropionate herbals; dicamba; glufosinate herbicides; herbicides which target the protox enzyme.

The plant or plant cell or plant part having the EPSPS sequence thereof can also be combined with at least one other trait to produce plants that further comprise a variety of desired trait combinations. For instance, the plant or plant cell or plant part having the EPSPS sequence or an active variant or fragment thereof may be stacked with polynucleotides encoding polypeptides having pesticidal and/or insecticidal activity, or a plant or plant cell or plant part having the EPSPS sequence thereof may be combined with a plant disease resistance gene.

These stacked combinations can be created by any method including, but not limited to, breeding plants by any conventional methodology, or genetic transformation.

### II. Methods of Use

### A. Methods of Generating Glyphosate Tolerant Plants

The terms “glyphosate tolerance” and “glyphosate resistance” are used interchangeably herein.

### I. Introducing

Various methods can be used to introduce a sequence of interest into a host cell, plant or plant part. The methods of the disclosure do not depend on a particular method for introducing a sequence into an organism or a plant or plant part, only that the polynucleotide or polypeptides gains access to the interior of at least one cell of the organism or the plant. Methods for introducing polynucleotide or polypeptide into various organisms, including plants, are known in the art including, but not limited to, stable transformation methods, transient transformation methods, and virus-mediated methods.

In specific embodiments, the EPSPS sequences thereof can be provided to a plant using a variety of transient transformation methods. Such transient transformation methods include, but are not limited to, the introduction of the EPSPS protein or active variants and fragments thereof directly into the plant. Such methods include, for example, microinjection or particle bombardment.

In other embodiments, the EPSPS polynucleotide disclosed herein or active variants thereof may be introduced into plants by contacting plants with a virus or viral nucleic acids. Generally, such methods involve incorporating a nucleotide construct of the disclosure within a DNA or RNA molecule.

Method are known in the art for the targeted insertion of a polynucleotide at a specific location in the plant genome. In one embodiment, the insertion of the polynucleotide at a desired genomic location is achieved using a site-specific recombination system.

The cells that have been transformed may be grown into plants in accordance with conventional ways. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as transgenic seed) having a polynucleotide disclosed herein, for example, as part of an expression cassette, stably incorporated into their genome.

Transformed plant cells which are derived by plant transformation techniques, including those discussed above, can be cultured to regenerate a whole plant which possesses the transformed genotype to glyphosate or a glyphosate analog.

One of skill will recognize that after the expression cassette containing the EPSPS gene is incorporated in transgenic plants and confirmed to be operable, it can be introduced into other plants by sexual crossing. Any of a number of standard breeding techniques can be used, depending upon the species to be crossed.

In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise V403A, wherein the amino acid mutation position corresponds to the amino acid position set forth in SEQ ID NO:1 and generating a glyphosate tolerant plant that comprising in its genome the recombinant DNA construct. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise G172A, P177S and/or V403A, wherein each amino acid mutation position corresponds to the amino acid position set forth in SEQ ID NO:1 and generating a glyphosate tolerant plant that comprising in its genome the recombinant DNA construct. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise T173I, P177S and/or V403A, wherein each amino acid mutation position corresponds to the amino acid position set forth in SEQ ID NO:1 and generating a glyphosate tolerant plant that comprising in its genome the recombinant DNA construct. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise T173I, P177T and/or V403A, wherein each amino acid mutation position corresponds to the amino acid position set forth in SEQ ID NO:1 and generating a glyphosate tolerant plant that comprising in its genome the recombinant DNA construct. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise G172A, P177S, and/or V403A, and at least one amino acid mutations selected from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO: 1 and generating a glyphosate tolerant plant that comprising in its genome the recombinant DNA construct.

In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise A70V, G172A, P177S, V403 and/orR514S mutation. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise A73T, G172A, P177S and/or V403A mutation. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise A73T, E86G, G172A, P177S and/or V403A mutation. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise A145G, G172A, P177S, V403A, T442S and/or D472E mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise G172A, P177S, V403A and/or F502V mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise G172A, P177S, Y252F and/or V403A mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise E137D, G172A, P177S, L253M and/or V403A mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise G172A, P177S, K399N, V403A and/or T466S mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise A73G, L134F, G172A, P177S, G265V, S374R and/or V403A mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise A73K, D114H, V136M, K161E, G172A, P177S, A260T and/or V403A mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise E163K, G172A, P177S, A216V, L253M, H389R and/or V403A mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise G172A, P177S, N246D and/or V403A mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise G172A, M175A, P177S, M258L, K356E, V403A and/or F512I mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise A73S, G172A, P177S, L214F and/or V403A mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise G172A, P177S, V208A, L264H, V403A and/or R514K mutations. In some embodiments, the methods comprise introducing by way of expressing in a regenerable plant cell a recombinant DNA construct comprising a polynucleotide operably linked to at least one regulatory sequence, wherein the polynucleotide encodes a plant EPSPS polypeptide that comprise V113M, G172A, P177S and/or V403A mutations.

### II. Modifying

In general, methods to modify or alter the host genomic DNA are known and available to one skilled in the art. For example, a pre-existing or endogenous EPSPS sequence in a host plant can be modified or altered in a site-specific fashion using one or more site-specific engineering systems. This includes altering the host DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” or engineered endonucleases such as meganucleases produced to modify plant genomes. Another site-directed engineering is through the use of zinc finger domain recognition coupled with the restriction properties of restriction enzyme. A transcription activator-like (TAL) effector-DNA modifying enzyme (TALE or TALEN) is also used to engineer changes in plant genome.

Site-specific modification of plant genomes can also be performed using CRISPR system (clustered regularly interspaced short palindromic repeats). For example, researchers can use the CRISPR system to generate DNA double strand breaks (DSBs) at specific sites in the plant genome, and use the error-prone non-homologous end joining (NHEJ) mechanism to generate the disclosed mutations. The specific mutations can also obtained through the precise base substitution under the guidance of an exogenous DNA donor (donor). Such base editing tools include but not limited to cytosine base editor (CBE) and/or adenine base editor (ABE). Or the prime editing system can also be used, the PBS (primer binding site) sequence in the pegRNA guides the mutations into the genome by using a reverse transcription template (RT template) containing the target editing sequence to generate these mutations.

An endogenous plant EPSPS gene in a plant cell may be modified to encode a glyphosate tolerant EPSPS protein that comprises V403A, the amino acid mutation position corresponds to the amino acid position set forth in SEQ ID NO: 1. An endogenous plant EPSPS gene in a plant cell can be modified to encode a glyphosate tolerant EPSPS protein that comprises G172A, P177S and/or V403A, wherein each amino acid mutation position corresponds to the amino acid position set forth in SEQ ID NO: 1. An endogenous plant EPSPS gene in a plant cell can be modified to encode a glyphosate tolerant EPSPS protein that comprises T173I, P177S and/or V403A wherein each amino acid mutation position corresponds to the amino acid position set forth in SEQ ID NO: 1. An endogenous plant EPSPS gene in a plant cell can be modified to encode a glyphosate tolerant EPSPS protein that comprises T173I, P177T and/or V403A wherein each amino acid mutation position corresponds to the amino acid position set forth in SEQ ID NO: 1. An endogenous plant EPSPS gene in a plant cell can be modified to encode a glyphosate tolerant EPSPS protein that comprises G192A, P177S, and/or V403A, and at least one amino acid mutations selected from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the amino acid position set forth in SEQ ID NO: 1.

An endogenous plant EPSPS gene in a plant cell may be modified to encode a glyphosate tolerant EPSPS protein that comprises an amino acid mutation that is analogous to V403A, wherein the amino acid mutation position corresponds to the analogous amino acid position set forth in SEQ ID NO: 1. An endogenous plant EPSPS gene in a plant cell can be modified to encode a glyphosate tolerant EPSPS protein that comprises an amino acid mutation that is analogous to G172A, an amino acid mutation that is analogous to P177S and/or an amino acid mutation that is analogous to V403A wherein each amino acid mutation position corresponds to the analogous amino acid position set forth in SEQ ID NO: 1. An endogenous plant EPSPS gene in a plant cell can be modified to encode a glyphosate tolerant EPSPS protein that comprises an amino acid mutation that is analogous to G172A, an amino acid mutation that is analogous to P177S, and/or an amino acid mutation that is analogous to V403A and at least one analogous amino acid mutations selected from the group consisting of: (1) A70V, (2) A73T/V/S/K/G, (3) V113M, (4) D114H, (5) L134F, (6) V136M, (7) E137D, (8) A145G, (9) K161E, (10) E163K, (11) M175A, (12) V208A, (13) L214F, (14) A216V, (15) Y252F, (16) L253M, (17) M258L, (18) A260T, (19) L264H, (20) G265V, (21) N316D, (22) K356E, (23) S374R, (24) H389R, (25) K399N, (26) T442S, (27) T466S, (28) D472E, (29) F502V, (30) F512I, and (31) R514S/K, wherein each amino acid mutation position corresponds to the analogous amino acid position set forth in SEQ ID NO: 1.

An endogenous plant EPSPS gene in a plant cell can be modified to encode a glyphosate tolerant EPSPS protein that comprises A70V, G172A, P177S, V403 and/orR514S mutation. An endogenous plant EPSPS gene in a plant cell can be modified to encode a glyphosate tolerant EPSPS protein that comprises A73T, G172A, P177S and/or V403A mutation. An endogenous plant EPSPS gene in a plant cell can be modified to encode a glyphosate tolerant EPSPS protein that comprises A73T, E86G, G172A, P177S and/or V403A mutation. An endogenous plant EPSPS gene in a plant cell can be modified to encode a glyphosate tolerant EPSPS protein that comprises A145G, G172A, P177S, V403A, T442S and/or D472E mutations. An endogenous plant EPSPS gene in a plant cell can be modified to encode a glyphosate tolerant EPSPS protein that comprises G172A, P177S, V403A and/or F502V mutations. An endogenous plant EPSPS gene in a plant cell can be modified to encode a glyphosate tolerant EPSPS protein that comprises G172A, P177S, Y252F and/or V403A mutations. An endogenous plant EPSPS gene in a plant cell can be modified to encode a glyphosate tolerant EPSPS protein that comprises E137D, G172A, P177S, L253M and/or V403A mutations. An endogenous plant EPSPS gene in a plant cell can be modified to encode a glyphosate tolerant EPSPS protein that comprises G172A, P177S, K399N, V403A and/or T466S mutations. An endogenous plant EPSPS gene in a plant cell can be modified to encode a glyphosate tolerant EPSPS protein that comprises A73G, L134F, G172A, P177S, G265V, S374R and/or V403A mutations. An endogenous plant EPSPS gene in a plant cell can be modified to encode a glyphosate tolerant EPSPS protein that comprises A73K, D114H, V136M, K161E, G172A, P177S, A260T and/or V403A mutations. An endogenous plant EPSPS gene in a plant cell can be modified to encode a glyphosate tolerant EPSPS protein that comprises E163K, G172A, P177S, A216V, L253M, H389R and/or V403A mutations. An endogenous plant EPSPS gene in a plant cell can be modified to encode a glyphosate tolerant EPSPS protein that comprises G172A, P177S, N246D and/or V403A mutations. An endogenous plant EPSPS gene in a plant cell can be modified to encode a glyphosate tolerant EPSPS protein that comprises G172A, M175A, P177S, M258L, K356E, V403A and/or F512I mutations. An endogenous plant EPSPS gene in a plant cell can be modified to encode a glyphosate tolerant EPSPS protein that comprises A73S, G172A, P177S, L214F and/or V403A mutations. An endogenous plant EPSPS gene in a plant cell can be modified to encode a glyphosate tolerant EPSPS protein that comprises G172A, P177S, V208A, L264H, V403A and/or R514K mutations. An endogenous plant EPSPS gene in a plant cell can be modified to encode a glyphosate tolerant EPSPS protein that comprises V113M, G172A, P177S and/or V403A mutations.

The endogenous plant EPSPS gene can be modified by a CRISPR/Cas guide RNA-mediated system, a Zn-finger nuclease-mediated system, a meganuclease-mediated system, an oligonucleobase-mediated system, TALEN, or any gene modification system known to one of ordinary skill in the art. Moreover, for the purpose herein, an endogenous plant EPSPS gene includes coding DNA and genomic DNA within and surrounding the coding DNA, such as for example, the promoter, intron, and terminator sequences.

### EXAMPLES

In the following Examples, it should be understood that these Examples, while indicating embodiments of the invention, are given by way of illustration only. From the above discussion and these Examples, one skilled in the art can make various changes and modifications of the invention to adapt it to various usages and conditions. Such modifications are also intended to fall within the scope of the appended embodiments.

### Example 1: Alignment of Rice EPSPS and Other Plant EPSPS Sequences

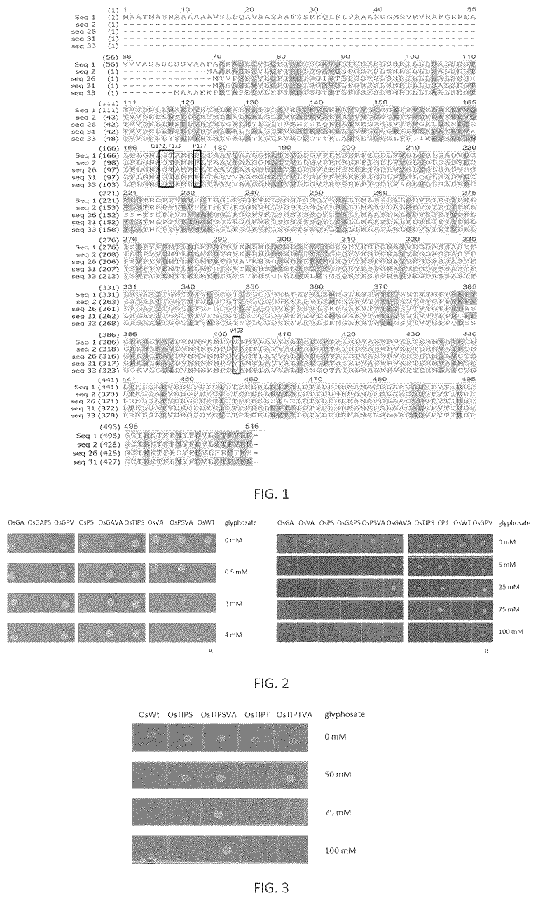

Using rice (Oryza sativa) full length EPSPS(SEQ ID NO: 1) as a reference, we aligned rice EPSPS(SEQ ID NO: 2), flax EPSPS(SEQ ID NO:26), wheat EPSPS(SEQ ID NO: 31) and soybean EPSPS(SEQ ID NO:33). The alignment result was shown in FIG. 1. It show the described mutation positions and the corresponding position in each plant species. All disclosed mutation positions were marked according to SEQ ID NO:1.

### Example 2: The Glyphosate Resistance Tests for Each Rice EPSPS Mutants on the Mutation Position of 403

The present inventors found V403 on rice EPSPS sequence plays a role in the sensitivity to glyphosate. The inventors performed saturation mutagenesis on the position 403 of the native rice EPSPS sequence to find out whether the mutation at this site can reduce the sensitivity of the rice EPSPS to glyphosate. Using NNK (where N represents adenine, thymine, guanine, and cytosine, and K represents thymine and thymine nucleotide) primers as the degenerate codon of the position to be mutated, the amino acid V on the position 403 of rice EPSPS polypeptide was mutated to the other 19 amino acids. The mutant PCR product was carried out using the construct pKED2 expressing the OsWt (SEQ ID NO:2) (FIG. 8) as the template, with V403 F61 as the forward primer and V403 R 61 as the reverse primer. The V403 F 61 primer sequence was AATGCCTGATNNKGCCATGACCC. The V403 R 61 primer sequence was TTGTTCATGTTGACATCAACAG.

The experiment steps and results were as following.

a) PCR system: According to the primer and template mixed PCR system corresponding to the target vector in the above table, the forward primer was 1.25 μl, the reverse primer was 1.25 μl, the template plasmid was 2 μl, the Q5 PCR master mix was 12.5 μl, and ddH₂O 18 μl.

b) PCR reaction condition:

| | | |
|---|---|---|
| Temperature | Time | Number of Cycles |

| 98° C. | 2 | min | |
|---|---|---|---|
| 98° C. | 15 | s | 30 cycles |
| 61° C. | 30 | s | |
| 72° C. | 2 min 30 s | | |
| 72° C. | 2 | min | |

c) Took out 5 μl of the above PCR product and added it to the buffer for agarose gel electrophoresis. The amplification was successful if a PCR product with a size of about 4.5 kb is obtained.

d) Using KLD enzyme mixture from NEB company to remove the template from the PCR product, and then the product was connected. The connection system was: KLD buffer 5 μl, KLD MIX 1 μl, PCR product 1 μl, ddH₂O 3 μl. After 5 minute ligation at room temperature, the PCR product was then transformed into E. coli.

e) The ligation product was transformed into the EPSPS-deficient E. coli Dh5α competent, and the EPSPS-deficient E. coli strain was constructed by the well-known Red homologous recombination method. (Guangxing Bai, Zhiwei Sun, Ying Huang, et al. Knockout of E. coli ClpP gene using Red recombination system[J]. Chinese Journal of Biochemistry and Molecular Biology (1): 45-48.) Both E. coli transformation method and the detection methods are conventionally known in the art. After resuscitation, the cells were washed twice with M9 medium, and then the washed cells were spread on M9 medium containing 0.2 mM glyphosate and cultured for 48 h. Sequencing every grown single colony (sequencing and its analysis is done by Tsingke Biological Technology Co., Ltd.), only V403A mutants was found to be resistant to 0.2 mM glyphosate.

### Example 3: The Comparison of the Glyphosate Resistance Among Rice EPSPS Mutants

Example 2 show the mutation V403A on rice EPSPS could affect the rice EPSPS sensitivity to the glyphosate. The inventor designed this experiment to test if any mutation on other position could also improve the mutated rice EPSPS sensitivity to the glyphosate.

The detailed experiment step and testing results were as following.

TABLE 2
The construct of Rice OsGA, OsPS, OsGAVA, OsPSVA, OsGAPS,
OsGPV, OsTIPS, OsTIPT, OsTIPSVA and OsTIPTVA mutants.

| Target | | | | Annealing |
|---|---|---|---|---|
| construct | Primer | Primer sequence | Template | Temperature |
| OsGA | G172A F | GGGAACGCTGcAACTGCAA | OsWt | 66° C. |
| | | TG | | |
| | G172A R | CAAGAAGAGTTGCACTTCC | | |
| OsPS | P177S F | TGCAATGCGACCATTGACA | OsWt | 66° C. |
| | | GC | | |
| | P177S R | GTTCCAGCGTTCCCCAAG | | |
| OsGAPS | GAPS F | atgcgatCATTGACAGCAGCCG | OsWt | 66° C. |
| | | TGA | | |
| | GAPS R | tgcagttgCAGCGTTCCCCAAG | | |
| | | AAGA | | |
| OsGAVA | G172AF | GGGAACGCTGcAACTGCAA | Os VA | 66° C. |
| | | TG | | |
| | G172A R | CAAGAAGAGTTGCACTTCC | | |
| OsPSVA | P177S F | TGCAATGCGACCATTGACA | Os VA | 66° C. |
| | | GC | | |
| | P177S R | GTTCCAGCGTTCCCCAAG | | |
| OsGPV | GAPS F | atgcgatCATTGACAGCAGCCG | Os VA | 66° C. |
| | | TGA | | |
| | GAPS R | tgcagttgCAGCGTTCCCCAAG | | |
| | | AAGA | | |
| Os TIPS | Os PS F | tgcgatCATTGACAGCAGCCG | OsWt | 66° C. |
| | 66 | TGA | | |
| | Os TI R | ttgcaaTTCCAGCGTTCCCCAA | | |
| | 66 | GA | | |
| Os TIPT | Os PT F | tgcgaaCATTGACAGCAGCCG | OsWt | 66° C. |
| | 66 | TGA | | |
| | Os TIR | ttgcaaTTCCAGCGTTCCCCAA | | |
| | 66 | GA | | |
| Os | Os PS F | tgcgatCATTGACAGCAGCCG | Os VA | 66° C. |
| TIPSVA | 66 | TGA | | |
| | Os TI R | ttgcaaTTCCAGCGTTCCCCAA | | |
| | 66 | GA | | |
| Os | Os PT F | tgcgaaCATTGACAGCAGCCG | Os VA | 66° C. |
| TIPTVA | 66 | TGA | | |
| | Os TI R | ttgcaaTTCCAGCGTTCCCCAA | | |
| | 66 | GA | | |

The detailed experiment steps as following:

### i: Obtain and Keep the Desired Rice EPSPS Mutant

a) PCR system: According to the primer and template mixed PCR system corresponding to the target vector in the above table, the forward primer was 1.25 μl, the reverse primer was 1.25 μl, the template plasmid was 2 μl, the Q5 PCR master mix was 12.5 μl, and ddH₂O was 18 μl.

| | | |
|---|---|---|
| Temperature | Time | The number of cycles |

| 98° | C. | 2 | min | |
|---|---|---|---|---|
| 98° | C. | 15 | s | 30 cycles |
| *° | C. | 30 | s | |
| 72° | C. | 2 min 30 s | | |
| 72° | C. | 2 | min | |

b) PCR reaction conditions were as following: the annealing temperature was set according to the annealing temperature corresponding to the target carrier in the above table.

*Set the annealing temperature as above table provided

c) Took out 5 μl of the obtained PCR product and added it to the loading buffer for agarose gel electrophoresis. A band with a size of about 4.5 kb was considered successful.

d) Used NEB's KLD enzyme mixture to remove the template and connected the PCR product. The connection system was 5 μl KLD buffer, 1 μl KLD MIX, 1 μl PCR product, and ddH₂O 3 μl. After ligating at room temperature for 5 min, transformed it into E. coli.

e) The ligation product was transformed into Dh5a competent E. coli lacking EPSPS. After resuscitation, the cells were washed twice with M9 medium, and then the washed cells were spread on M9 medium and cultured for 48 h. Sequenced the grown single colony (Tsingke Biological Technology Co., Ltd.), and selected the correct mutant to save for future use.

Pick the following mutants for testing:

OsWt represents the wild type rice EPSPS(SEQ ID NO:2). OsVA represents the mutated rice EPSPS with mutation V403A(SEQ ID NO:3). OsGA represents the mutated rice EPSPS with mutation G172A. (SEQ ID NO:7) OsPS represents the mutated rice EPSPS with mutation P177S. (SEQ ID NO:8) OsGAPS represents the mutated rice EPSPS with mutation G172A and P177S. (SEQ ID NO:9) OsGAVA represents the mutated rice EPSPS with mutation G172A and V403A(SEQ ID NO:4). OsPSVA represents the mutated rice EPSPS with mutation P177S and V403A(SEQ ID NO:5). OsGPV represents the mutated rice EPSPS with mutation G172A, P177S and V403A(SEQ ID NO:6). OsTIPS represents the mutated rice EPSPS with mutation T173I and P177S. (SEQ ID NO: 35). OsTIPT represents the mutated rice EPSPS with mutation T173I and P177T. (SEQ ID NO:36). OsTIPSVA represents the mutated rice EPSPS with mutation T173I, P177S and V403A. (SEQ ID NO:37). OsTIPTVA represents the mutated rice EPSPS with mutation T173I, P177T and V403A. (SEQ ID NO:38). CP4 represents the wild type EPSPS from Agrobacterium tumefaciens. (SEQ ID NO:39)

### ii: The Glyphosate Resistance Comparison Among Rice EPSPS Mutants

Each product obtained from the above steps were transformed into E. coli lacking EPSPS. By detecting the growth of the transformed Escherichia coli on M9 medium containing different glyphosate concentrations (0 mM, 0.5 mM, 2 mM, 4 mM, 5 mM, 25 mM, 75 mM, 100 mM), the 1 mM glyphosate here was equivalent to 169 mg/ml glyphosate acid, the glyphosate resistance of rice EPSPS mutants was verified. The results were shown in FIG. 2.

On the medium containing 0 mM glyphosate, the strains expressing all other genes can grow normally except that the E. coli lacking EPSPS containing OsGAPS cannot grow. This result indicated that except OsGAPS mutants, all other rice EPSPS mutants can be expressed in E. coli to compensate the function of original EPSPS in E. coli.

The OsGA, OsPS and OsVA EPSPS mutants can grow on 0.5 mM glyphosate medium, but the OsWt cannot grow, indicating that either G172A, P177S or V403A single mutation can improve the glyphosate resistance of the rice EPSPS mutant. Here, V403A is a newly discovered mutation to improve EPSPS glyphosate tolerance.

FIG. 2 show that the OsGAVA can grow on 75 mM glyphosate medium or any medium with lesser than 75 mM glyphosate and the OsPSVA can grow on 4 mM glyphosate medium or any medium with lesser than 4 mM glyphosate. The V403A mutation combing with either G172A or P177S mutation can improve the glyphosate resistance of the rice EPSPS mutant.

FIG. 2 further show the OsGPV can grow on 100 mM glyphosate medium while the glyphosate resistance was comparable to the glyphosate resistance from CP4 EPSPS. The additional mutation of V403A can improve the glyphosate resistance of OsGAVA or OsGAPS.

FIG. 3 further show the OsTIPTVA can grow on 100 mM and OsTIPTVA can grow on 75 mM glyphosate medium. The additional mutation of V403 A can improve the glyphosate resistance of OsTIPS or OsTIPT.

### Example 4: The Glyphosate Resistance for Flax EPSPS Mutants

### i: Obtain the Flax EPSPS Mutant

The flax (Linum usitatissimum) EPSPS mutant sequence(SEQ ID NO: 27) was synthesized and cloned into the pKED2 vector to obtain the vector LuVA. The vector LuWt was created after introducing A403V mutation by PCR.

The detailed experiment steps as following:

| Target | | primer | Temp- | Annealing |
|---|---|---|---|---|
| Vector | primer name | sequence | late | temperature |
| LuWt | Lu 403V_F 63 | ATGCCAGACGt | LuVA | 63º C. |
| | | TGCGATGACT | | |
| | Lu 403V_R 63 | TTTGTTCATGT | | |
| | | TAACATCAACA | | |
| | | GC | | |

a) PCR system: According to the primer and template mixed PCR system corresponding to the target vector in the above table, the forward primer was 1.25 μl, the reverse primer was 1.25 μl, the template plasmid was 2 μl, the Q5 PCR master mix was 12.5 μl, and ddH₂O was 18 μl.

| | | |
|---|---|---|
| Temperature | Time | The number of cycles |

| 98° C. | 2 | min | |
|---|---|---|---|
| 98° C. | 15 | s | 30 cycles |
| 63° C. | 30 | s | |
| 72° C. | 2 min 30 s | | |
| 72° C. | 2 | min | |

b) PCR reaction conditions were as follows: the annealing temperature was set according to the annealing temperature corresponding to the target carrier in the above table.

c) Took out 5 μl of the obtained PCR product and added it to the loading buffer for agarose gel electrophoresis. A band with a size of about 4.5 kb was considered successful.

d) Used NEB's KLD enzyme mixture to remove the template and then connected the PCR product. The connection system was 5 μl KLD buffer, 1 μl KLD MIX, 1 μl PCR product, and ddH₂O 3 μl. After ligating at room temperature for 5 min, transformed it into E. coli.

e) The ligation product was transformed into Dh5a competent E. coli lacking EPSPS. After resuscitation, the cells were washed twice with M9 medium, and then the washed cells were spread on M9 medium and cultured for 48 h. Sequenced the grown single colony (Tsingke Biological Technology Co., Ltd.), and selected the correct mutant to save for future use.

ii: Resistance Test of Flax EPSPS Mutant and Comparison with Wild Type

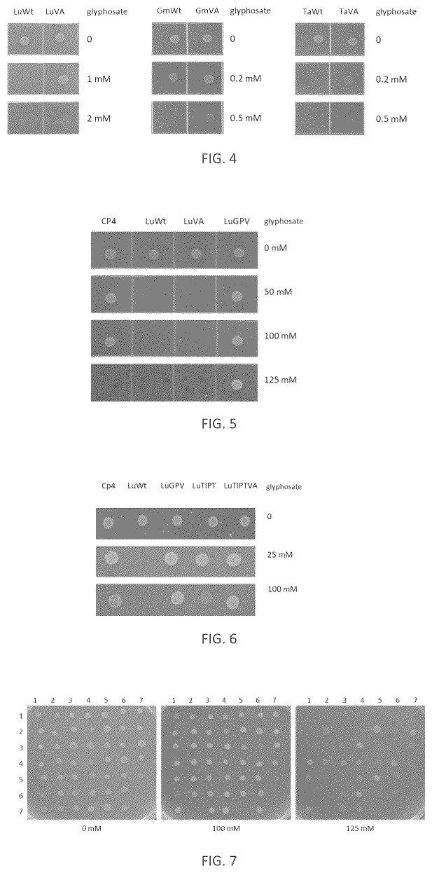

The resistance results were shown in FIG. 4: LuWt(SEQ ID NO:26) represents the wild-type flax EPSPS gene, and LuVA (SEQ ID NO:27) represents the V403A single-point mutant of flax EPSPS.

With the presence of 1 mM and 2 mM glyphosate, the growth status of LuVA was significantly better than that of LuWt, indicating that the V403A mutation can increase the glyphosate resistance of flax EPSPS.

### Example 5: The Glyphosate Resistance for the Soybean EPSPS Mutant

### i: Obtain the Desired Soybean EPSPS Mutant

According to the wild-type sequence information of soybean (Glycine max) EPSPS (SEQ ID NO:36), the gene was synthesized and inserted into the vector pKED2 to obtain the vector GmWt. The V403 site of GmWt was mutated to A by PCR, and the vector GmVA expressing the soybean EPSPS mutant protein containing the V403A mutation was obtained. The specific experimental steps were as following:

| Targt | Primer | Primer | | Annealing |
|---|---|---|---|---|
| Vector | Name | Sequence | Template | Temperature |
| GmVA | Gm EP | ATGCCAGATGcTGC | GmWt | 63° C. |
| | VA F 63 | CATGACT | | |
| | Gm EP | CTTGTTCATATTG | | |
| | VAR 63 | ACATCAATGCC | | |

a) PCR system: According to the primer and template mixed PCR system corresponding to the target vector in the above table, the forward primer was 1.25 μl, the reverse primer was 1.25 μl, the template plasmid was 2 μl, the Q5 PCR master mix was 12.5 μl, and ddH₂O was 18 μl.

| | | |
|---|---|---|
| Temperature | Time | The number of cycles |

| 98° C. | 2 | min | |
|---|---|---|---|
| 98° C. | 15 | s | 30 cycles |
| 63° C. | 30 | s | |
| 72° C. | 2 min 30 s | | |
| 72° C. | 2 | min | |

b) PCR reaction conditions were as follows: the annealing temperature was set according to the annealing temperature corresponding to the target carrier in the above table.

c) Took out 5 μl of the obtained PCR product and added it to the loading buffer for agarose gel electrophoresis. A band with a size of about 4.5 kb was considered successful.

d) Used NEB's KLD enzyme mixture to remove the template and connected the PCR product. The connection system was 5 μl KLD buffer, 1 μl KLD MIX, 1 μl PCR product, and ddH₂O 3 μl. After ligating at room temperature for 5 min, transformed it into E. coli.

e) The ligation product was transformed into Dh5a competent E. coli lacking EPSPS. After resuscitation, the cells were washed twice with M9 medium, and then the washed cells were spread on M9 medium and cultured for 48 h. Sequenced the grown single colony (Tsingke Biological Technology Co., Ltd.), and selected the correct mutant to save for future use.

ii: Resistance Test of Soybean EPSPS Mutant and Comparison with Wild Type

The resistance results were shown in FIG. 4: GmWt (SEQ ID NO:33) represent the wild-type soybean EPSPS gene, and GmVA(SEQ ID NO:34) represent the V403A single-point mutant of soybean EPSPS.

Under the pressure of 0.5 mM glyphosate, the growth status of GmVA was significantly higher than that of GmWt, indicating that the V403A mutation can increase the glyphosate resistance of soybean EPSPS.

### Example 6: The Glyphosate Resistance for the Wheat EPSPS Mutant

### i: Experimental Steps for Obtaining Wheat EPSPS Mutants

The gene was synthesized according to the V403A mutant sequence information (SEQ ID NO:32) of the EPSPS gene on chromosome 7 of the wheat A genome and inserted into the vector pKED2 to obtain the wheat (Triticum vulgare) EPSPS mutant expression vector TaVA containing the V403A mutation. The A403 site of TaVA was mutated back to V by PCR, and a TaWt vector expressing the EPSPS wild-type gene on chromosome 7 of wheat A genome was obtained.

The specific experimental steps were as following:

| Target | Primer | | | Annealing |
|---|---|---|---|---|
| Vector | Name | Primer Sequence | Template | Temperature |
| TaWt | Ta 403V | ATGCCAGATGtCGCG | TaVA | 64° C. |
| | F 64 | ATGACT | | |
| | Ta 403 | TTTGTTCATGTTGAC | | |
| | VR 64 | ATCAACAGC | | |

a) PCR system: According to the primer and template mixed PCR system corresponding to the target vector in the above table, the forward primer was 1.25 μl, the reverse primer was 1.25 μl, the template plasmid was 2 μl, the Q5 PCR master mix was 12.5 μl, and ddH₂O was 18 μl.

| | | |
|---|---|---|
| Temperature | Time | The Number of Cycles |

| 98° C. | 2 | min | |
|---|---|---|---|
| 98° C. | 15 | s | 30 cycles |
| 64° C. | 30 | s | |
| 72° C. | 2 min 30 s | | |
| 72° C. | 2 | min | |

b) PCR reaction conditions were as follows: the annealing temperature was set according to the annealing temperature corresponding to the target carrier in the above table.

c) Took out 5 μl of the obtained PCR product and added it to the loading buffer for agarose gel electrophoresis. A band with a size of about 4.5 kb was considered successful.

d) Used NEB's KLD enzyme mixture to remove the template and then connected the PCR product. The connection system was 5 μl KLD buffer, 1 μl KLD MIX, 1 μl PCR product, and ddH₂O 3 μl. After ligating at room temperature for 5 min, transformed it into E. coli.

e) The ligation product was transformed into Dh5a competent E. coli lacking EPSPS. After resuscitation, the cells were washed twice with M9 medium, and then the washed cells were spread on M9 medium and cultured for 48 h. Sequenced the grown single colony (Tsingke Biological Technology Co., Ltd.), and selected the correct mutant to save for future use.

ii: Resistance Test of Wheat EPSPS Mutant and Comparison with Wild Type

The resistance results were shown in FIG. 4: TaWt(SEQ ID NO:31) represents the wild-type EPSPS gene on chromosome 7 of the wheat A genome, and TaVA(SEQ ID NO:32) represents the V403A single-point mutant of wheat EPSPS.

Under the pressure of 0.2 mM glyphosate, the growth state of TaVA was significantly higher than that of TaWt, indicating that the V403A mutation can increase the glyphosate resistance of wheat EPSPS.

### Example 7: Resistance Identification of Flax EPSPS Mutants LuGPV, LuTIPT, LuTIPTVA

The G172 position and P177 position of LuVA were mutated into A and S at the same time by PCR, and a 3-point mutant LuGPV was obtained.

The T173 and P177 positions of LuWT were simultaneously mutated to I and T by PCR, and a 3-point mutant LuTIPT was obtained.

The T173 and P177 positions of LuVA were simultaneously mutated to I and T by PCR, and a 3-point mutant LuTIPTVA was obtained.

The specific experimental steps were as following:

| Target | | Primer | | Annealing |
|---|---|---|---|---|
| Vector | Primer | Sequence | Template | Temperature |
| LuGPV | Lu 172A | atgcgttcgCTGACA | LuVA | 63° C. |
| | 177S_F 61 | GCTGCTGTAACAG | | |
| | Lu 172A | agctgttgcAGCATT | | |
| | 177S_R 61 | TCCAAGGAAAAGTTC | | |
| LuTIPT | Lu PT | tgcgtaCGCTGACAG | LuVA | 64° C. |
| VA | F 64 | CTGCTGTAA | | |
| | Lu TI | tagctaTTCCAGCAT | | |
| | R 64 | TTCCAAGGAAAAG | | |
| LuTIPT | Lu PT | tgcgtaCGCTGACAG | LuWT | 64° C. |
| | F 64 | CTGCTGTAA | | |
| | Lu TI | tagctaTTCCAGCAT | | |
| | R 64 | TTCCAAGGAAAAG | | |

a) PCR system: According to the primer and template mixed PCR system corresponding to the target vector in the above table, the forward primer was 1.25 μl, the reverse primer was 1.25 μl, the template plasmid was 2 μl, the Q5 PCR master mix was 12.5 μl, and ddH₂O was 18 μl.

| | | |
|---|---|---|
| Temperature | Time | The Number of Cycles |

| 98° C. | 2 | min | |
|---|---|---|---|
| 98° C. | 15 | s | 30 cycles |
| 63° C./64° C. | 30 | s | |
| 72° C. | 2 min 30 s | | |
| 72° C. | 2 | min | |

b) PCR reaction conditions were as follows: the annealing temperature was set according to the annealing temperature corresponding to the target carrier in the above table.

c) Took out 5 μl of the obtained PCR product and added it to the loading buffer for agarose gel electrophoresis. A band with a size of about 4.5 kb was considered successful.

d) Used NEB's KLD enzyme mixture to remove the template and connected the PCR product. The connection system was 5 μl KLD buffer, 1 μl KLD MIX, 1 μl PCR product, and ddH₂O 3 μl. After ligating at room temperature for 5 min, transformed it into E. coli.

e) The ligation product was transformed into Dh5a competent E. coli lacking EPSPS. After resuscitation, the cells were washed twice with M9 medium, and then the washed cells were spread on M9 medium and cultured for 48 h. Sequenced the grown single colony (Tsingke Biological Technology Co., Ltd.), and selected the correct mutant to save for future use.

ii: Resistance Test of EPSPS Mutant of Flax and Comparison with Wild Type

The resistance results were shown in FIG. 5: LuWt(SEQ ID NO:26) represents the EPSPS2 gene of flax, LuGPV (SEQ ID NO:30) represents the flax G172A+P177S+V403A three-point mutant. The resistance results were shown in FIG. 6: LuTIPT (SEQ ID NO:28) was the flax T173I+P177S double-point mutants, LuTIPTVA (SEQ ID NO:29) was flax T173I+P177T+V403A three-point mutants.

The results showed that the GPV mutant of flax had strong glyphosate resistance. From the colony saturation under 100 mM glyphosate pressure, the resistance of LuGPV was higher than CP4. The resistance of LuTIPTVA was higher than that of LuTIPT, indicating that V403A can also significantly increase the resistance level of the mutant in the flax EPSPS gene.

### Example 7: The Resistance Test for Other Rice Mutants

In order to further improve the glyphosate resistance and develop highly resistant rice EPSPS mutants that can meet commercial production requirements, we used OsGPV as a template to perform random mutations, with high-concentration glyphosate screening, and to identify EPSPS mutant that can improve the glyphosate resistance.

i. Obtain the Rice Mutants

### 1) Error-Prone PCR

We used OsGPV(SEQ ID NO:6) as a template to perform error-prone PCR. (With GeneMorph II Random Mutagenesis Kit, #200550, Agilent Technologies).

Reaction requirement:

| | | |
|---|---|---|
| 10× Mutazyme II reaction buffer | 5 | μl |
| dNTP mix (200 μM each final) | 1 | μl |
| EP OsF | 0.5 | μl |
| EP OsR | 0.5 | μl |
| Mutazyme II DNA polymerase (2.5 U/μl) | 1 | μl |
| ddH₂O | X | μl |
| OsGPV | 300 | ng |
| Total | 50 | μl |

Primer sequence as following:

EP OsF: tgtgaattcatggcggcgaaggcggaggagatc
EP OsR: gatcctgcaggtcagttcctgacgaaagtgc

Procedure of PCR reaction:

| | | |
|---|---|---|
| Temperature | Time | The Number of Cycles |

| | | | |
|---|---|---|---|
| 95° C. | 2 | min | |
| 95° C. | 30 | s | 40 cycles |
| 60° C. | 30 | s | |
| 72° C. | 90 | s | |
| 72° C. | 10 | min | |

### 2) Performed PCR Product Recovery and Purification on the Obtained PCR Product

### 3) Recovered Product and Vector Backbone Digestion

a) The recovered product was digested with EcoRI and SbfI, and then ligated to the pKED2 vector after the same double digestion with T₄ DNA ligase to obtain the ligation product to obtain the vector carrying the rice EPSPS gene mutant.

b) Transformed the above vector into Escherichia coli DH5α with EPSPS gene knocked out. The transformation steps were as follows: add the above ligation product to 50 μl E. coli DH5α competent cells with EPSPS gene knocked out, mix well and incubate on ice for 30 min, then after a heat shock at 42° C. for 90 s, immediately place it in an ice bath on ice for 2 min after taking it out. After the ice bath, add 500 μl of LB liquid medium and resuscitate at 37° C. at 180 r/min for 45 min. The transformed Escherichia coli was a mutant library expressing rice EPSPS mutant genes obtained on the template basis of OsGPV.

4) Resistance screening

5) Centrifuged the transformed product obtained above at 3000 rpm for 5 min, discarded the supernatant, then resuspended it in 1 ml M9 liquid medium, added 200 μl M9 liquid medium to resuspend and inoculated it on an M9 plate containing 75 mM glyphosate. Incubated at 37 degrees for 24 hours.

Pick and saved the single colonies grown in the above screening plates, and conduct further resistance identification and sequencing

ii. Resistance test of rice mutants. In this example, the wild-type rice EPSPS gene OsWT, the most widely used commercial glyphosate-resistant gene CP4EPSPS, the rice mutantOsTIPS, which had the same mutation type with commercial glyphosate-resistant corn GA21, and the rice mutant OsGPV with the highly glyphosate-resistant rice EPSPS mutant gene obtained in this study were transformed as control, and the newly obtained rice EPSPS mutants were transformed as the experimental group, and the glyphosate resistance among the control and the mutants were compared. The monoclonal bacteria of the control group and the experimental group were respectively inoculated into a medium containing 0, 100 mM and 125 mM glyphosate, and the growth of colonies in each group was observed.

The order of Samples (from left to right):
1^{st} Row: 1-8, 1-13, 1-19, 1-24, 1-27, 1-37, 1-38
2^{nd} Row: 1-42, 1-68, 1-88, 1-93, 1-98, 1-125, 1-139
3^{rd} Row: 1-193, 1-150, 1-217, 1-246, 1-250, 1-265, 1-386
4^{th} Row: 1-411, 1-547, 1-552, 1-599, 1-614, 1-631, 1-130
5^{th} Row: 2-5, 2-43, 2-45, 2-46, 2-47, 2-63, 2-121
6^{th} Row: 2-114, 2-133, 3-8, 3-25, 3-27, 3-30, 3-32
7^{th} Row: 3-33, OsTIPS, 3-36, CP4, Os Wt, Os GPV

In the above results, under the pressure of 100 mM glyphosate, except for 3-32 and OsTIPS that did not grow, all other experimental and control samples were able to tolerate/resist the glyphosate, but the growth status of the colonies was inconsistent. Based on the colony growth status under 125 mM glyphosate pressure, the colony growth status of the 16 mutants (1-68 (SEQ ID NO:10), 1-98(SEQ ID NO:11), 1-139(SEQ ID NO:12), 1-246(SEQ ID NO:13), 1-386(SEQ ID NO:14), 1-411(SEQ ID NO:15), 1-547(SEQ ID NO:16), 1-552(SEQ ID NO:17), 1-599(SEQ ID NO:18), (SEQ ID NO:19), 2-5(SEQ ID NO:20), 2-46(SEQ ID NO:21), 2-47(SEQ ID NO:22), (SEQ ID NO:23), 3-33(SEQ ID NO:24), 3-36(SEQ ID NO:25) were significantly higher than OsGPV in colony growth status, and some of the mutants were significantly more resistant than CP4, showing increased resistance. The colony growth status of the other 16 mutant showed decreased resistance than OsGPV. The other 12 mutants had no significant difference in resistance from OsGPVin FIG. 7.

### Example 8: Transgenic Rice Plant's Resistance to Glyphosate Herbicide

The rice seed used in this example is RICE KENG XIANG DA008-169, which is publicly available.

1. Obtaining the expression vector.

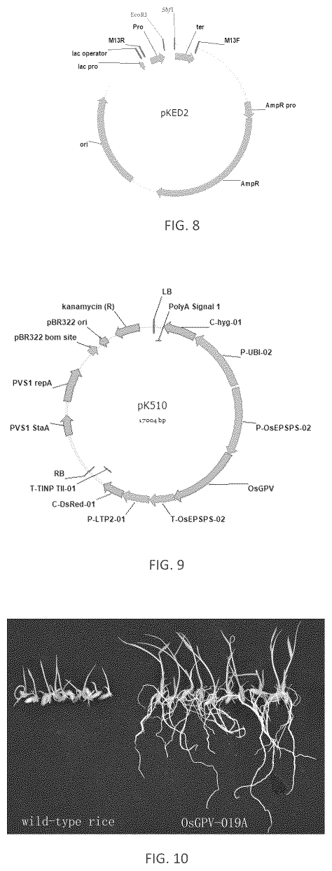

Nucleic acid sequence encoding OsGPV mutants (comprising G172A, P177S and V403A) (SEQ ID NO:6) were inserted into the plant expression vector according to the conventional methods to obtain the pk510 vector expressing the OsGPV gene. (see FIG. 9)

2. Transgenic experiments in rice callus.

1) The transgenic vector pk510 was introduced into Agrobacterium EHA105 to obtain recombinant Agrobacterium.

2) One to five days before the transformation experiment, recombinant Agrobacterium was inoculated on YEB solid medium containing 50 mg/L kanamycin and 15 mg/L rifampicin. The medium was sealed in the dish with 3M tape; kept at temperature at 28° C. in an inverted dish orientation and cultured it in dark for one to five days.

3) The bacteria was scraped at the “Z”-shaped tail with the inoculation ring, and gently suspended in 100 μM NB liquid medium containing 2 mg/L 2,4-dichlorophenoxyacetic acid to make the concentration of the bacteria liquid reach approximately O.D. 600=0.1, and then reserve the make Agrobacterium infection solution.

4) After removing the rice seed coat, the rice seed was placed in a triangular flask, washed 3 times with sterile water. 75% alcohol was added to immerse the seeds, shaken gently for 1 min, and the alcohol discarded. Then, 2.5% sodium chlorate aqueous solution was added and the solution shaken at 150-170 rpm for 25 min. Next, sterile water was added and gently shake for 5-8 times, then pour out water. The seeds were inoculated on a callus induction medium and cultured in the dark at 30° C. for 4-6 weeks and rice callus obtained.

5) The rice callus obtained in step 4 was soaked in the Agrobacterium infection solution for 10 minutes, and then placed on a co-culture medium covered with two layers of sterilized filter paper, and cultured in the dark at 22° C. for 3 days.

6) The rice callus obtained in step 5 was taken and put it on the recovery medium, and cultivated for 4-7 days at 30° C.

7) The rice callus obtained in step 6 was taken and placed on the hygromycin selection medium, and cultured at 30° C. for 2 weeks.

8) The rice callus obtained in step 7 was taken and placed on the hygromycin selection medium, and cultured at 30° C. for 2 weeks.

9) The vigorously growing callus was transferred to Differentiation medium ingredients and cultured at 30° C. for 20-30 days. Plants were produced from yellowish or green callus.

10) Robust regenerated green seedlings larger than 3 cm in height were selected, transferred to rooting medium, and cultivated for 7-14 days to obtain rice TO seedlings.

### Medium Ingredient:

Co-culture medium ingredients: NB solid medium containing 2 mg/L 2,4-dichlorophenoxyacetic acid
recovery medium ingredients: NB solid medium containing 200 mg/L timentin
hygromycin selection medium ingredients: NB solid medium containing 30-50 mg/L hygromycin
Differentiation medium ingredients: NB solid medium containing 0.5 g/L glutamine, 0.5 g/L proline, 2 mg/L kanamycin and 0.2 mg/L α-naphthoacetic acid.
Rooting medium ingredients: MS solid medium containing 0.5 g/L glutamine, 0.5 g/L proline, 0.2 mg/L α-naphthaleneacetic acid.

Transgenic rice TO seedlings were cultivated in an artificial climate chamber until harvest, and T1 generation rice seeds were obtained for the glyphosate resistance test.

2. Identification of glyphosate resistance in T1 generation transgenic rice.

Transgenic rice TO seedlings were cultivated in an artificial climate chamber until harvest, and T1 generation rice seeds were obtained for the glyphosate resistance test.

1) T1 generation seeds that were positive for transgene under ultraviolet light were selected. Since the plant expression vector was constructed by expressing OsGPV and the DsRed gene that can express red fluorescent protein in tandem, the T1 generation seeds can be differentiated by exposure to ultraviolet light. The seeds of red light were the seeds which contain both DsRed and OsGPV vectors.

2) Accelerating germination: 20 seeds from each of the non-transgenic control seeds and T1 transgenic seeds containing the OsGPV gene were taken, and soaked in clean water for 4 hours to make the seeds fully absorb water. Then the seeds were transferred to a petri dish with two layers of filter paper at the bottom, and added a small amount of clean water to it to keep it moist. The amount of water should not overfill the seeds.

3) After culturing for 2 days following the above method, the seeds were transferred to a petri dish (diameter 9 cm) with two layers of filter paper at the bottom, and a small amount of 5 ml of 100 ppm glyphosate aqueous solution was added (Roundup, adding 41% isopropylamine salt solution to dilute 10,000 times) and cultivated for 10 days.

4) Ten rice seedlings were randomly picked and photos were taken, see FIG. 10.

In FIG. 10, it show that the growth of the wild-type rice as control was significantly inhibited in the 100 ppm glyphosate solution, and the root length was also significantly inhibited. In the subsequent observations, all the wild type died and the root rotted. The transgenic rice containing OsGPV mutants was not significantly affected by glyphosate herbicide and continued to grow, indicating that the rice plants with the OsGPV mutants was the glyphosate tolerate/resistant.

1. A polynucleotide encoding a plant EPSP synthase (EPSPS) polypeptide, wherein the plant EPSPS polypeptide comprises amino acid V mutating to amino acid A, wherein the mutation location is the location of the amino acid V in the EPSPS amino acid sequence of the plant corresponding to the location of V403 listed in SEQ ID NO:1.

2. (canceled)

3. The polynucleotide of claim 1, wherein the plant EPSPS polypeptide comprises G172A and V403A amino acid mutations,
wherein the plant EPSPS polypeptide comprises P177S and V403A amino acid mutations,
wherein the plant EPSPS polypeptide comprises G172A, P177S, and V403A amino acid mutations,
wherein the plant EPSPS polypeptide comprises T173I, P177S, and V403A amino acid mutations,
wherein the plant EPSPS polypeptide comprises T173I, P177T, and V403A amino acid mutations,
wherein the polynucleotide encodes a plant EPSPS polypeptide that comprises A70V, G172A, P177S, V403A, and R514S,
wherein the polynucleotide encodes a plant EPSPS polypeptide that comprises A73T, G172A, P177S, and V403A,
wherein the polynucleotide encodes a plant EPSPS polypeptide that comprises A73T, E86G, G172A, P177S, and V403A,
wherein the polynucleotide encodes a plant EPSPS polypeptide that comprises A145G, G172A, P177S, V403A, T442S, and D472E,
wherein the polynucleotide encodes a plant EPSPS polypeptide that comprises G172A, P177S, V403A and F502V,
wherein the polynucleotide encodes a plant EPSPS polypeptide that comprises G172A, P177S, Y252F and V403A,
wherein the polynucleotide encodes a plant EPSPS polypeptide that comprises E137D, G172A, P177S, L253M and V403A,
wherein the polynucleotide encodes a plant EPSPS polypeptide that comprises G172A, P177S, K399N, V403A and T466S,
wherein the polynucleotide encodes a plant EPSPS polypeptide that comprises A73G, L134G, G172A, P177S, G265V, S374R and V403A,
wherein the polynucleotide encodes a plant EPSPS polypeptide that comprises A73K, D114H, V136M, K161E, G172A, P177S, A260T and V403A,
wherein the polynucleotide encodes a plant EPSPS polypeptide that comprises E163K, G172A, P177S, A216V, L253M, H389R and V403A,
wherein the polynucleotide encodes a plant EPSPS polypeptide that comprises G172A, P177S, N247D and V403A,
wherein the polynucleotide encodes a plant EPSPS polypeptide that comprises G172A, M175A, P177S, M258L, K356E, V403A and F512I,
wherein the polynucleotide encodes a plant EPSPS polypeptide that comprises A73S, G172A, P177S, L214F and V403A,
wherein the polynucleotide encodes a plant EPSPS polypeptide that comprises G172A, P177S, V208A, L264H, V403A and R514K, or
wherein the polynucleotide encodes a plant EPSPS polypeptide that comprises V113M, G172A, P177S and V403A.

4. The polynucleotide of claim 3, wherein the plant EPSPS polypeptide is shown as any one of SEO ID NO: 3-25, 27-30, 32, and 34-38.

5. The polynucleotide of claim 4, wherein the plant EPSPS polypeptide is shown as SEQ ID NO: 6.

6-24. (canceled)

25. A recombinant DNA construct comprising the polynucleotide of claim 1.

26-27. (canceled)

28. A plant comprising in its genome the polynucleotide of claim 1.

29. The plant of claim 28, wherein said plant is corn, rice, wheat, barley, oat, sorghum, millet, flax, hemp, kentucky bluegrass, tall fescue, creeping bentgrass, Brassica sp., sugar beets, soybean, peas, alfalfa, tobacco, cotton, sunflower, tomato, potato, apple tree, grapevine, citrus trees, cucumber or petunia.

30. The plant of claim 29, wherein said plant is rice.

31-107. (canceled)


SEQUENCE LISTING (39 sequences)

SEQ ID NO: 1 - protein, 515 aa, Artificial Sequence
  This sequence is synthesized in lab.
MAATMASNAAAAAAVSLDQAVAASAAFSSRKQLRLPAAARGGMRVRVRARGRREAVVVASASSSSVAAPAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGTAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 2 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGTAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 3 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNGATAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 4 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 5 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNGATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 6 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 7 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRPLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 8 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGTAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 9 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 10 - protein, 447 aa, Artificial Sequence
  This sequence is synthesized in lab.
MVAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVSN

SEQ ID NO: 11 - protein, 447 aa, Artificial Sequence
  This sequence is synthesized in lab.
MAAKTEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 12 - protein, 447 aa, Artificial Sequence
  This sequence is synthesized in lab.
MAAKTEEIVLQPIREISGAVQLPGSKSLSNRILLLSALSEGTTVVDNLLNSEDVHYMLEALKALGLSVEADKVAKRAVVVGCGGKFPVGKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 13 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRGVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELSKLGASVEEGPDYCIITPPEKLNITAIDTYEDHRMAMAFSLAACADVPVTIRDPGCTRKTFPNYFDVLSTFVRN

SEQ ID NO: 14 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTVPNYFDVLSTFVRN

SEQ ID NO: 15 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQFLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 16 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVDADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYMSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 17 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNNMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 18 - protein, 447 aa, Artificial Sequence
  This sequence is synthesized in lab.
MAAKGEEIVLQPIREISGAVQLPGSKSLSNRILLLSALSEGTTVVDNLLNSEDVHYMLEALKALGFSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALVDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTRVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 19 - protein, 447 aa, Artificial Sequence
  This sequence is synthesized in lab.
MAAKKEEIVLQPIREISGAVQLPGSKSLSNRILLLSALSEGTTVVHNLLNSEDVHYMLEALKALGLSMEADKVAKRAVVVGCGGKFPVEKDAEEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMATPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 20 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEKVQLFLGNAATAMRSLTAAVTAAGGNATYVLDGVPRMRERPIGDLVVGLKQLGVDVDCFLGTECPPVRVKGIGGLPGGKVKLSGSISSQYMSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKRLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 21 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGDAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 22 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAARSLTAAVTAAGGNATYVLDGVPRMRERPIGDLVVGLKQLGADVDCFLGTECPPVRVKGIGGLPGGKVKLSGSISSQYLSALLLAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVEFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTIVRN

SEQ ID NO: 23 - protein, 447 aa, Artificial Sequence
  This sequence is synthesized in lab.
MAAKSEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQFGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 24 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAATAMRSLTAAVTAAGGNATYVLDGVPRMRERPIGDLAVGLKQLGADVDCFLGTECPPVRVKGIGGLPGGKVKLSGSISSQYLSALLMAAPLAHGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVKN

SEQ ID NO: 25 - protein, 447 aa, Artificial Sequence
  This sequence is synthesized in lab.
MAAKAEEIVLQPIREISGAVQLPGSKSLSNRILLLSALSEGTTVMDNLLNSEDVHYMLEALKALGLSVEADKVAKRAVVVGCGGKFPVEKDAKEEVQLFLGNAATAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 26 - protein, 445 aa, Artificial Sequence
  This sequence is synthesized in lab.
MTVPEEIVLQPIKDISGIVTLPGSKSLSNRILLLAALSEGKTVVDNLLNSDDVHYMLGALKTLGLNVEHSSEQKRAIVEGRGGVFPVGKLGKNDIELFLGNAGTAMRPLTAAVTAAGGNSSYILDGVPRMRERPIGDLVVGLKQLGADVSCSSTSCPPVHVNAKGGLPGGKVKLSGSISSQYLTALLMAAPLALGDVEIEIVDKLISVPYVDMTLKLMERFGVAVEHSGSWDRFFVKGGQKYKSPGNAYVEGDASSASYFLAGAAITGGTITVEGCGTSSLQGDVKFAEVLEKMGAKVTWTETSVTVTGPPRDASGKKHLRAVDVNMNKMPDVAMTLAVVALYADGPTAIRDVASWRVKETERMIAVCTELRKLGATVEEGPDYCIITPPEKLSIAEIDTYDDHRMAMAFSLAACADVPVTIRDPGCTKKTFPDYFEVLERYTKH

SEQ ID NO: 27 - protein, 445 aa, Artificial Sequence
  This sequence is synthesized in lab.
MTVPEEIVLQPIKDISGIVTLPGSKSLSNRILLLAALSEGKTVVDNLLNSDDVHYMLGALKTLGLNVEHSSEQKRAIVEGRGGVFPVGKLGKNDIELFLGNAGTAMRPLTAAVTAAGGNSSYILDGVPRMRERPIGDLVVGLKQLGADVSCSSTSCPPVHVNAKGGLPGGKVKLSGSISSQYLTALLMAAPLALGDVEIEIVDKLISVPYVDMTLKLMERFGVAVEHSGSWDRFFVKGGQKYKSPGNAYVEGDASSASYFLAGAAITGGTITVEGCGTSSLQGDVKFAEVLEKMGAKVTWTETSVTVTGPPRDASGKKHLRAVDVNMNKMPDAAMTLAVVALYADGPTAIRDVASWRVKETERMIAVCTELRKLGATVEEGPDYCIITPPEKLSIAEIDTYDDHRMAMAFSLAACADVPVTIRDPGCTKKTFPDYFEVLERYTKH

SEQ ID NO: 28 - protein, 445 aa, Artificial Sequence
  This sequence is synthesized in lab.
MTVPEEIVLQPIKDISGIVTLPGSKSLSNRILLLAALSEGKTVVDNLLNSDDVHYMLGALKTLGLNVEHSSEQKRAIVEGRGGVFPVGKLGKNDIELFLGNAGIAMRTLTAAVTAAGGNSSYILDGVPRMRERPIGDLVVGLKQLGADVSCSSTSCPPVHVNAKGGLPGGKVKLSGSISSQYLTALLMAAPLALGDVEIEIVDKLISVPYVDMTLKLMERFGVAVEHSGSWDRFFVKGGQKYKSPGNAYVEGDASSASYFLAGAAITGGTITVEGCGTSSLQGDVKFAEVLEKMGAKVTWTETSVTVTGPPRDASGKKHLRAVDVNMNKMPDVAMTLAVVALYADGPTAIRDVASWRVKETERMIAVCTELRKLGATVEEGPDYCIITPPEKLSIAEIDTYDDHRMAMAFSLAACADVPVTIRDPGCTKKTFPDYFEVLERYTKH

SEQ ID NO: 29 - protein, 445 aa, Artificial Sequence
  This sequence is synthesized in lab.
MTVPEEIVLQPIKDISGIVTLPGSKSLSNRILLLAALSEGKTVVDNLLNSDDVHYMLGALKTLGLNVEHSSEQKRAIVEGRGGVFPVGKLGKNDIELFLGNAGIAMRTLTAAVTAAGGNSSYILDGVPRMRERPIGDLVVGLKQLGADVSCSSTSCPPVHVNAKGGLPGGKVKLSGSISSQYLTALLMAAPLALGDVEIEIVDKLISVPYVDMTLKLMERFGVAVEHSGSWDRFFVKGGQKYKSPGNAYVEGDASSASYFLAGAAITGGTITVEGCGTSSLQGDVKFAEVLEKMGAKVTWTETSVTVTGPPRDASGKKHLRAVDVNMNKMPDAAMTLAVVALYADGPTAIRDVASWRVKETERMIAVCTELRKLGATVEEGPDYCIITPPEKLSIAEIDTYDDHRMAMAFSLAACADVPVTIRDPGCTKKTFPDYFEVLERYTKH

SEQ ID NO: 30 - protein, 445 aa, Artificial Sequence
  This sequence is synthesized in lab.
MTVPEEIVLQPIKDISGIVTLPGSKSLSNRILLLAALSEGKTVVDNLLNSDDVHYMLGALKTLGLNVEHSSEQKRAIVEGRGGVFPVGKLGKNDIELFLGNAATAMRSLTAAVTAAGGNSSYILDGVPRMRERPIGDLVVGLKQLGADVSCSSTSCPPVHVNAKGGLPGGKVKLSGSISSQYLTALLMAAPLALGDVEIEIVDKLISVPYVDMTLKLMERFGVAVEHSGSWDRFFVKGGQKYKSPGNAYVEGDASSASYFLAGAAITGGTITVEGCGTSSLQGDVKFAEVLEKMGAKVTWTETSVTVTGPPRDASGKKHLRAVDVNMNKMPDAAMTLAVVALYADGPTAIRDVASWRVKETERMIAVCTELRKLGATVEEGPDYCIITPPEKLSIAEIDTYDDHRMAMAFSLAACADVPVTIRDPGCTKKTFPDYFEVLERYTKH

SEQ ID NO: 31 - protein, 446 aa, Artificial Sequence
  This sequence is synthesized in lab.
MAGAEEVVLQPIREISGAVQLPGSKSLSNRILLLSALSEGTTVVDNLLNSEDVHYMLEALEALGLSVEADKVAKRAVVVGCGGRFPVEKDAKEEVKLFLGNAGTAMRPLTAAVVAAGGNATYVLDGVPRMRERPIGDLVVGLQQLGADVDCFLGTNCPPVRINGKGGLPGGKVKLSGSISSQYLSSLLMAAPLALEDVEIEIIDKLISVPYVEMTLKLMEHFGVTAEHSDSWDRFYIKGGQKYKSPGNAYVEGDASSASYFLAGAAITGGTVTVEGCGTTSLQGDVKFAEVLEMMGAKVTWTDTSVTVTGPPRQPFGRKHLKAVDVNMNKMPDVAMTLAVVALFADGPTAIRDVASWRVKETERMVAIRTELTKLGATVEEGPDYCIITPPEKLNITAIDTYDDHRMAMAFSLAACAEVPVTIRDPGCTRKTFPNYFDVLSTFVKN

SEQ ID NO: 32 - protein, 446 aa, Artificial Sequence
  This sequence is synthesized in lab.
MAGAEEVVLQPIREISGAVQLPGSKSLSNRILLLSALSEGTTVVDNLLNSEDVHYMLEALEALGLSVEADKVAKRAVVVGCGGRFPVEKDAKEEVKLFLGNAGTAMRPLTAAVVAAGGNATYVLDGVPRMRERPIGDLVVGLQQLGADVDCFLGTNCPPVRINGKGGLPGGKVKLSGSISSQYLSSLLMAAPLALEDVEIEIIDKLISVPYVEMTLKLMEHFGVTAEHSDSWDRFYIKGGQKYKSPGNAYVEGDASSASYFLAGAAITGGTVTVEGCGTTSLQGDVKFAEVLEMMGAKVTWTDTSVTVTGPPRQPFGRKHLKAVDVNMNKMPDAAMTLAVVALFADGPTAIRDVASWRVKETERMVAIRTELTKLGATVEEGPDYCIITPPEKLNITAIDTYDDHRMAMAFSLAACAEVPVTIRDPGCTRKTFPNYFDVLSTFVKN

SEQ ID NO: 33 - protein, 452 aa, Artificial Sequence
  This sequence is synthesized in lab.
MAAAEKPSTAPEIVLEPIKDISGTITLPGSKSLSNRILLLAALSEGTTVVDNLLYSEDIHYMLGALRTLGLRVEDDQTTKQAIVEGCGGLFPTIKESKDEINLFLGNAGTAMRPLTAAVVAAGGNASYVLDGVPRMRERPIGDLVAGLKQLGADVDCFLGTNCPPVRVNGKGGLPGGKVKLSGSISSQYLTALLMAAPLALGDVEIEIVDKLISVPYVEMTLKLMERFGVSVEHSGNWDKFLVHGGQKYKSPGNAFVEGDASSASYFLAGAAVTGGTITVNGCGTNSLQGDVKFAEVLEKMGAKVTWSENSVTVTGPPQDSSGQKVLQGIDVNMNKMPDVAMTLAVVALFANGQTAIRDVASWRVKETERMIAICTELRKLGATVEEGPDYCVITPPEKLNVTAIDTYDDHRMAMAFSLAACGDVPVTIKDPGCTRKTFPDYFEVLERFTRH

SEQ ID NO: 34 - protein, 452 aa, Artificial Sequence
  This sequence is synthesized in lab.
MAAAEKPSTAPEIVLEPIKDISGTITLPGSKSLSNRILLLAALSEGTTVVDNLLYSEDIHYMLGALRTLGLRVEDDQTTKQAIVEGCGGLFPTIKESKDEINLFLGNAGTAMRPLTAAVVAAGGNASYVLDGVPRMRERPIGDLVAGLKQLGADVDCFLGTNCPPVRVNGKGGLPGGKVKLSGSISSQYLTALLMAAPLALGDVEIEIVDKLISVPYVEMTLKLMERFGVSVEHSGNWDKFLVHGGQKYKSPGNAFVEGDASSASYFLAGAAVTGGTITVNGCGTNSLQGDVKFAEVLEKMGAKVTWSENSVTVTGPPQDSSGQKVLQGIDVNMNKMPDAAMTLAVVALFANGQTAIRDVASWRVKETERMIAICTELRKLGATVEEGPDYCVITPPEKLNVTAIDTYDDHRMAMAFSLAACGDVPVTIKDPGCTRKTFPDYFEVLERFTRH

SEQ ID NO: 35 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGIAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 36 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGIAMRTLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDVAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 37 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGIAMRSLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 38 - protein, 447 aa, Artificial Sequence
  This sequence is synthesized in lab.
MAAKAEEIVLQPIREISGAVQLPGSKSLSNRILLLSALSEGTTVVDNLLNSEDVHYMLEALKALGLSVEADKVAKRAVVVGCGGKFPVEKDAKEEVQLFLGNAGIAMRTLTAAVTAAGGNATYVLDGVPRMRERPIGDLVVGLKQLGADVDCFLGTECPPVRVKGIGGLPGGKVKLSGSISSQYLSALLMAAPLALGDVEIEIIDKLISIPYVEMTLRLMERFGVKAEHSDSWDRFYIKGGQKYKSPGNAYVEGDASSASYFLAGAAITGGTVTVQGCGTTSLQGDVKFAEVLEMMGAKVTWTDTSVTVTGPPREPYGKKHLKAVDVNMNKMPDAAMTLAVVALFADGPTAIRDVASWRVKETERMVAIRTELTKLGASVEEGPDYCIITPPEKLNITAIDTYDDHRMAMAFSLAACADVPVTIRDPGCTRKTFPNYFDVLSTFVRN

SEQ ID NO: 39 - protein, 459 aa, Artificial Sequence
  This sequence is synthesized in lab.
MDPGMSHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA